SEQUENCE LISTING (45 sequences)

SEQ ID NO: 1 - protein, 1102 aa, Homo sapiens
MFQLPVNNLGSLRKARKTVKKILSDIGLEYCKEHIEDFKQFEPNDFYLKNTTWEDVGLWDPSLTKNQDYRTKPFCCSACPFSSKFFSAYKSHFRNVHSEDFENRILLNCPYCTFNADKKTLETHIKIFHAPNASAPSSSLSTFKDKNKNDGLKPKQADSVEQAVYYCKKCTYRDPLYEIVRKHIYREHFQHVAAPYIAKAGEKSLNGAVPLGSNAREESSIHCKRCLFMPKSYEALVQHVIEDHERIGYQVTAMIGHTNVVVPRSKPLMLIAPKPQDKKSMGLPPRIGSLASGNVRSLPSQQMVNRLSIPKPNLNSTGVNMMSSVHLQQNNYGVKSVGQGYSVGQSMRLGLGGNAPVSIPQQSQSVKQLLPSGNGRSYGLGSEQRSQAPARYSLQSANASSLSSGQLKSPSLSQSQASRVLGQSSSKPAAAATGPPPGNTSSTQKWKICTICNELFPENVYSVHFEKEHKAEKVPAVANYIMKIHNFTSKCLYCNRYLPTDTLLNHMLIHGLSCPYCRSTFNDVEKMAAHMRMVHIDEEMGPKTDSTLSFDLTLQQGSHTNIHLLVTTYNLRDAPAESVAYHAQNNPPVPPKPQPKVQEKADIPVKSSPQAAVPYKKDVGKTLCPLCFSILKGPISDALAHHLRERHQVIQTVHPVEKKLTYKCIHCLGVYTSNMTASTITLHLVHCRGVGKTQNGQDKTNAPSRLNQSPSLAPVKRTYEQMEFPLLKKRKLDDDSDSPSFFEEKPEEPVVLALDPKGHEDDSYEARKSFLTKYFNKQPYPTRREIEKLAASLWLWKSDIASHFSNKRKKCVRDCEKYKPGVLLGFNMKELNKVKHEMDFDAEWLFENHDEKDSRVNASKTADKKLNLGKEDDSSSDSFENLEEESNESGSPFDPVFEVEPKISNDNPEEHVLKVIPEDASESEEKLDQKEDGSKYETIHLTEEPTKLMHNASDSEVDQDDVVEWKDGASPSESGPGSQQVSDFEDNTCEMKPGTWSDESSQSEDARSSKPAAKKKATMQGDREQLKWKNSSYGKVEGFWSKDQSQWKNASENDERLSNPQIEWQNSTIDSEDGEQFDNMTDGVAEPMHGSLAGVKLSSQQA

SEQ ID NO: 2 - protein, 63 aa, Artificial Sequence
  Peptide
EARKSFLTKYFNKQPYPTRREIEKLAASLWLWKSDIASHFSNKRKKCVRDCEKYKPGVLLGFN

SEQ ID NO: 3 - protein, 1131 aa, Homo sapiens
MFQIPVENLDNIRKVRKKVKGILVDIGLDSCKELLKDLKGFDPGEKYFHNTSWGDVSLWEPSGKKVRYRTKPYCCGLCKYSTKVLTSFKNHLHRYHEDEIDQELVIPCPNCVFASQPKVVGRHFRMFHAPVRKVQNYTVNILGETKSSRSDVISFTCLKCNFSNTLYYSMKKHVLVAHFHYLINSYFGLRTEEMGEQPKTNDTVSIEKIPPPDKYYCKKCNANASSQDALMYHILTSDIHRDLENKLRSVISEHIKRTGLLKQTHIAPKPAAHLAAPANGSAPSAPAQPPCFHLALPQNSPSPAAGQPVTVAQGAPGSLTHSPPAAGQSHMTLVSSPLPVGQNSLTLQPPAPQPVFLSHGVPLHQSVNPPVLPLSQPVGPVNKSVGTSVLPINQTVRPGVLPLTQPVGPINRPVGPGVLPVSPSVTPGVLQAVSPGVLSVSRAVPSGVLPAGQMTPAGQMTPAGVIPGQTATSGVLPTGQMVQSGVLPVGQTAPSRVLPPGQTAPLRVISAGQVVPSGLLSPNQTVSSSAVVPVNQGVNSGVLQLSQPVVSGVLPVGQPVRPGVLQLNQTVGTNILPVNQPVRPGASQNTTFLTSGSILRQLIPTGKQVNGIPTYTLAPVSVTLPVPPGGLATVAPPQMPIQLLPSGAAAPMAGSMPGMPSPPVLVNAAQSVFVQASSSAADTNQVLKQAKQWKTCPVCNELFPSNVYQVHMEVAHKHSESKSGEKLEPEKLAACAPFLKWMREKTVRCLSCKCLVSEEELIHHLLMHGLGCLFCPCTFHDIKGLSEHSRNRHLGKKKLPMDYSNRGFQLDVDANGNLLFPHLDFITILPKEKLGEREVYLAILAGIHSKSLVPVYVKVRPQAEGTPGSTGKRVSTCPFCFGPFVTTEAYELHLKERHHIMPTVHTVLKSPAFKCIHCCGVYTGNMTLAAIAVHLVRCRSAPKDSSSDLQAQPGFIHNSELLLVSGEVMHDSSFSVKRKLPDGHLGAEDQRHGEEQPPILNADAAPGPEKVTSVVPFKRQRNESRTEGPIVKDEALQILALDPKKYEGRSYEEKKQFLKDYFHKKPYPSKKEIELLSSLFWVWKIDVASFFGKRRYICMKAIKNHKPSVLLGFDMSELKNVKHRLNFEYEP

SEQ ID NO: 4 - protein, 63 aa, Artificial Sequence
  Synthetic core site peptide
EEKKQFLKDYFHKKPYPSKKEIELLSSLFWVWKIDVASFFGKRRYICMKAIKNHKPSVLLGFD

SEQ ID NO: 5 - protein, 63 aa, Artificial Sequence; identical to SEQ ID NO: 4
  Synthetic core site peptide

SEQ ID NO: 6 - protein, 5 aa, Artificial Sequence
  Synthetic core site peptide
PGVLL

SEQ ID NO: 7 - protein, 4 aa, Artificial Sequence
  Synthetic core site peptide
ARKS

SEQ ID NO: 8 - protein, 16 aa, Artificial Sequence
  Synthetic core site peptide
SDIASHFSNKRKKCVR

SEQ ID NO: 9 - protein, 18 aa, Artificial Sequence
  Synthetic core site peptide
KRTYEQMEFPLLKKRKLD

SEQ ID NO: 10 - protein, 5 aa, Artificial Sequence
  Synthetic core site peptide
PSVLL

SEQ ID NO: 11 - protein, 4 aa, Artificial Sequence
  Synthetic core site peptide
EKKQ

SEQ ID NO: 12 - protein, 16 aa, Artificial Sequence
  Synthetic core site peptide
IDVASFFGKRRYICMK

SEQ ID NO: 13 - protein, 9 aa, Artificial Sequence
  Synthetic core site peptide
SALLRSIPA

SEQ ID NO: 14 - protein, 8 aa, Artificial Sequence
  Synthetic core site peptide
NAPVSIPQ

SEQ ID NO: 15 - protein, 11 aa, Artificial Sequence
  Synthetic core site peptide
XSALLRSIPAX

SEQ ID NO: 16 - protein, 14 aa, Artificial Sequence
  Synthetic core site peptide
VLGGGSALLRSIPA

SEQ ID NO: 17 - protein, 19 aa, Artificial Sequence
  Synthetic core site peptide
VEEGIVLGGGSALLRSIPA

SEQ ID NO: 18 - protein, 13 aa, Artificial Sequence
  Synthetic core site peptide
LGGGSALLRSIPA

SEQ ID NO: 19 - protein, 12 aa, Artificial Sequence
  Synthetic core site peptide
GGGSALLRSIPA

SEQ ID NO: 20 - protein, 11 aa, Artificial Sequence
  Synthetic core site peptide
GGSALLRSIPA

SEQ ID NO: 21 - protein, 10 aa, Artificial Sequence
  Synthetic core site peptide
GSALLRSIPA

SEQ ID NO: 22 - protein, 10 aa, Artificial Sequence
  Synthetic core site peptide
XNAPVSIPQX

SEQ ID NO: 23 - protein, 10 aa, Artificial Sequence
  Synthetic core site peptide
GGNAPVSIPQ

SEQ ID NO: 24 - protein, 13 aa, Artificial Sequence
  Synthetic core site peptide
LGGNAPVSIPQQS

SEQ ID NO: 25 - protein, 15 aa, Artificial Sequence
  Synthetic core site peptide
LGLGGNAPVSIPQQS

SEQ ID NO: 26 - protein, 18 aa, Artificial Sequence
  Synthetic core site peptide
SVRLGLGGNAPVSIPQQS

SEQ ID NO: 27 - DNA, 25 bp, Artificial Sequence
  synthetic antisense polynucleotide
tagtcctgcaacatttgagaaacca

SEQ ID NO: 28 - DNA, 25 bp, Artificial Sequence
  synthetic antisense polynucleotide
ttgttcactgggagttgaaacattc

SEQ ID NO: 29 - DNA, 25 bp, Artificial Sequence
  synthetic antisense polynucleotide
cactggaatctgatacattttgctc

SEQ ID NO: 30 - DNA, 25 bp, Artificial Sequence
  synthetic antisense polynucleotide
accttttactggaaactggtacatg

SEQ ID NO: 31 - DNA, 25 bp, Artificial Sequence
  synthetic negative control polynucleotide
cctcttacctcagttacaatttata

SEQ ID NO: 32 - DNA, 21 bp, Artificial Sequence
  synthetic mouse ADNP primer polynucleotide
acgaaaaatcaggactatcgg

SEQ ID NO: 33 - DNA, 21 bp, Artificial Sequence
  synthetic mouse ADNP primer polynucleotide
ggacattccggaaatgacttt

SEQ ID NO: 34 - DNA, 20 bp, Artificial Sequence
  synthetic mouse ADNP2 primer polynucleotide
ggaaagaaagcgagataccg

SEQ ID NO: 35 - DNA, 20 bp, Artificial Sequence
  synthetic mouse ADNP2 primer polynucleotide
tcctggtcagcctcatcttc

SEQ ID NO: 36 - DNA, 18 bp, Artificial Sequence
  synthetic mouse Hbb-b1 primer polynucleotide
gcaggctgctggttgtct

SEQ ID NO: 37 - DNA, 18 bp, Artificial Sequence
  synthetic mouse Hbb-b1 primer polynucleotide
catgggccttcactttgg

SEQ ID NO: 38 - DNA, 19 bp, Artificial Sequence
  synthetic mouse Slc41a primer polynucleotide
tgcttgtgggactgtccat

SEQ ID NO: 39 - DNA, 20 bp, Artificial Sequence
  synthetic mouse Slc41a primer polynucleotide
aagagctggatgccactgag

SEQ ID NO: 40 - DNA, 20 bp, Artificial Sequence
  synthetic mouse c-kit primer polynucleotide
caaaggaaatgcacgactgc

SEQ ID NO: 41 - DNA, 20 bp, Artificial Sequence
  synthetic mouse c-kit primer polynucleotide
tcccataggaccagacatca

SEQ ID NO: 42 - DNA, 21 bp, Artificial Sequence
  synthetic mouse Smarca4 primer polynucleotide
gcaggatgaggaggaagatga

SEQ ID NO: 43 - DNA, 20 bp, Artificial Sequence
  synthetic mouse Smarca4 primer polynucleotide
gcgcatgaagaggtcaaact

SEQ ID NO: 44 - DNA, 20 bp, Artificial Sequence
  synthetic mouse 18S ribosomal primer polynucleotide
cctgcggcttaatttgactc

SEQ ID NO: 45 - DNA, 20 bp, Artificial Sequence
  synthetic mouse 18S ribosomal primer polynucleotide
aactaagaacggccatgcac

(19) United States
(12) Patent Application Publication
(10) Pub. No.: US 2013/0053320 A1
(43) Pub. Date: Feb. 28, 2013

(54) TREATMENT OF ANEMIA BY ADNP AND ADNF POLYPEPTIDES

(71) Applicants: Illana Gozes, Ramat-Hasharon (IL); Efrat Dresner, Holon (IL)

(72) Inventors: Illana Gozes, Ramat-Hasharon (IL); Efrat Dresner, Holon (IL)

(73) Assignee: Ramot at Tel-Aviv University, Ltd., Tel Aviv (IL)

(21) Appl. No.: 13/578,362

(22) PCT Filed: Feb. 13, 2011
(86) PCT No.: PCT/IL2011/000152

§ 371 Date: Oct. 29, 2012

Related U.S. Application Data
(60) Provisional application No. 61/304,076, filed on Feb. 12, 2010.

(51) Int. Cl. A61K 38/17; A61P 7/06

(52) U.S. Cl. 514/13.5

(57) ABSTRACT

This invention relates to the use of ADNP, ADNP2, and ADNF polypeptides in the treatment of anemia and related conditions. Compositions of the ADNP, ADNP2, or ADNF polypeptides for such use are disclosed. Also provided are methods for concentrating or extracting ADNP, ADNP2, or ADNF polypeptides from dairy products and the use of dairy products in the context of treating anemia.

### FIELD AND BACKGROUND OF THE INVENTION

Activity-dependent neuroprotective protein (ADNP) is essential for mouse embryonic brain formation. Its family member, ADNP2, is associated with cell survival but remains to be fully evaluated for its role during embryogenesis. The present inventors discovered that ADNP and ADNP2 are required during zebrafish embryonic development for proper maturation of the erythroid lineage, but not for mesodermal specification to the hematopoietic lineage, myeloid differentiation or primitive erythroid development. Silencing of ADNP or ADNP2 negatively affected erythrocyte number and hemoglobin synthesis. Similarly, silencing of either ADNP or ADNP2 in mouse erythroleukemia (MEL) cells, an established model for erythropoiesis, resulted in impaired erythroid differentiation, which was associated with reduced expression of Brg1, an ADNP-interacting chromatin-remodeling protein required for erythropoiesis. Exogenous RNA encoding ADNP or ADNP2 rescued the undifferentiated phenotype of MEL cells. Through their discovery of an ancestral evolutionary-conserved role for the ADNP protein family in the maturation and differentiation processes of the erythroid lineage, the present inventors for the first time have indicated the use of ADNP and related polypeptides for treating anemic conditions such as one caused by an impaired erythropoiesis.

The highly conserved activity-dependent neuroprotective protein (ADNP) and ADNP2 constitute a protein family, containing zinc finger motifs and a homeobox profile (Bassan et al., 1999, J Neurochem 72, 1283-1293; Zamostiano et al., 2001, J Biol Chem 276, 708-714). ADNP was originally cloned from the P19 teratocarcinoma cell line induced to differentiate into neuroglial cells (Bassan et al., 1999, supra). In this cell line ADNP was found to associate with chromatin upon neuro-differentiation, and immunoprecipitation analyses identified binding between ADNP and heterochromatin protein 1α (Mandel et al., 2007, J Biol Chem 276, 708-714). In addition, ADNP directly interacts with Brg1, a member of the SWI/SNF chromatin remodeling complex (Mandel and Gozes, 2007, J Biol Chem 282, 34448-34456).

During embryogenesis, pronounced ADNP expression was found in the developing mouse brain, and ADNP was shown as an essential protein for brain development. ADNP-knockout mice exhibited marked growth retardation, failure to complete axial rotation and defects in the closure of the cranial neural tube, and did not survive beyond embryonic day 9.5 (Pinhasov et al., 2003, Brain Res Dev 144, 83-90). At this developmental point, ADNP was shown to regulate >400 genes, including genes associated with organogenesis, neurogenesis and heart development (Mandel et al., 2007, supra).

ADNP2 is an ADNP homologue with 33% identity and 46% similarity (Zamostiano et al., 2001, supra). Initial characterization of ADNP2 revealed an expression pattern that resembles that of ADNP; in the developing mouse embryo, high ADNP2 expression was found in the brain and was sustained throughout embryogenesis and adulthood. In addition, ADNP2 transcripts were found to be ubiquitously expressed in multiple mouse tissues (Kushnir et al., 2008, J Neurochem 105, 537-545), implying involvement in the functioning of many organs. Similar to ADNP that has been shown to protect neuronal-like cells (Steingart and Gozes, 2006, Mol Cell Endocrinol 252, 148-153), ADNP2 was associated with cell survival and protected P19 cells from oxidative damage (Kushnir et al., 2008, supra). ADNP and ADNP2 may be similarly regulated, as a high correlation was found between their mRNA expression patterns (Dresner et al., 2010, Eur Neuropsychopharmacol, EPUB ahead of print).

The present inventors set out to elucidate ADNP and ADNP2 developmental function. Considering the high homology between ADNP2 and ADNP, the inventors hypothesized that ADNP2 may also have an important role during development. The zebrafish model was chosen as a model system. ADNP-knockout mice die in uteri at very early stages, precluding the characterization of their phenotype at high resolution. The zebrafish is an excellent vertebrate model to study development; the external fertilization, the rapid development and the optical transparency of the embryos (Davidson and Zon, 2004, Oncogene 23, 7233-7246) present great advantages over the mouse in-uteri development. The results from zebrafish and from mouse erythroleukemia (MEL) cells reveal a novel evolutionary-conserved role for the ADNP protein family in erythropoiesis, and suggest that this function might be mediated at least in part through regulation of Brg1, a member of the SWI/SNF chromatin remodeling complex.

The inventors' further studies showed that ADNP polypeptide was detectable in the milk by an anti-ADNP antibody and such ADNP presence directly correlated with globin production by MEL cells upon exposure to dairy products. These results indicate that ADNP and related polypeptides are effective in promoting erythropoiesis and are therefore useful for treating anemia and related conditions.

### BRIEF SUMMARY OF THE INVENTION

This invention relates to the use of an ADNP or ADNF polypeptide in the treatment of anemia and other conditions involving inadequate red blood cells. The present invention also relates to the manufacture of medicaments, methods of formulation, methods of isolating the ADNP or ADNF polypeptide, and uses of the polypeptide.

In one aspect, this invention provides a method for treating anemia. The method comprises administering a therapeutically effective amount of an ADNP polypeptide to a subject in need thereof. The ADNP polypeptide is: (a) a full length ADNP polypeptide (SEQ ID NO:1); (b) a fragment of the full length ADNP polypeptide comprising an active core site of PGVLL (SEQ ID NO:6), ARKS (SEQ ID NO:7), SDIASHFSNKRKKCVR (SEQ ID NO:8), or KRTYEQMEFPLLKKRKLD (SEQ ID NO:9); or (c) a polypeptide comprising the full length ADNP polypeptide of (a) or the fragment of (b) and having up to about 40 amino acids at either or both of the N-terminus and the C-terminus.

In some cases, the fragment in (b) described above comprises an active core site of E A R K S F L T K Y F N K Q P Y P T R R E I E K L A A S L W L W K S D I A S H F S N K R K K C V R D C E K Y K P G V L L G F N (SEQ ID NO:2). In some certain embodiments of the method, the ADNP polypeptide is a full length ADNP polypeptide, having the amino acid sequence of SEQ ID NO:1. In other cases, the polypeptide in (c) has up to about 40 or 20 amino acids at either or both of the N-terminus and the C-terminus. In one example, the ADNP polypeptide consists of SEQ ID NO:2. In various embodiments or examples described above, the active core site of the ADNP polypeptide comprises at least one D-amino acid. In some cases, all amino acids in a core site may be D-amino acids.

In another aspect, the present invention provides method for treating anemia by administering a therapeutically effective amount of an ADNP2 polypeptide to a subject in need thereof. The ADNP2 polypeptide is: (a) a full length ADNP2 polypeptide (SEQ ID NO:3); (b) a fragment of the full length ADNP2 polypeptide comprising an active core site of PSVLL (SEQ ID NO:10), EKKQ (SEQ ID NO:11), or IDVASFFGKRRYICMK (SEQ ID NO:12); or (c) a polypeptide comprising the full length ADNP2 polypeptide of (a) or the fragment of (b) and having up to about 40 amino acids at either or both of the N-terminus and the C-terminus.

In some cases, the fragment described above in (b) comprises an active core site of E E K K Q F L K D Y F H K K P Y P S K K E I E L L S S L F W V W K I D V A S F F G K R R Y I C M K A I K N H K P S V L L G F D (SEQ ID NO:4). In certain embodiments of the method, the ADNP2 polypeptide is the full length ADNP2 polypeptide, having the amino acid sequence of SEQ ID NO:3. In some cases, the polypeptide described above in (c) has up to about 40 or 20 amino acids at either or both of the N-terminus and the C-terminus. In one example, the ADNP2 polypeptide consists of SEQ ID NO:4. In various embodiments or examples described above, the active core site of the ADNP2 polypeptide comprises at least one D-amino acid. In some cases, all amino acids in a core site may be D-amino acids.

In the above described methods, the ADNP (also known as ADNP1) polypeptide or the ADNP2 polypeptide (sometimes collectedly referred to in this application as “ADNP polypeptide” for convenience) may be administered intranasally, orally, intravenously, or subcutaneously. One example of means of ADNP polypeptide administration is by injection, e.g., by intravenous injection. Another possible route for ADNP polypeptide administration is by oral administration, such as by ingesting an effective amount of a dairy product, due to the presence of ADNP polypeptides in the milk. The ADNP polypeptides may be administered with a dairy product as well.

The present inventor discovered that ADNP polypeptides are present in milk or dairy products. As such, in a third aspect, the present invention provides a method for isolating an ADNP polypeptide. The method includes these steps: (1) contacting, under conditions permitting antigen-antibody binding, a dairy product in an aqueous solution with a solid substrate comprising an immobilized antibody that specifically binds the ADNP polypeptide (which may be an ADNP1 or ADNP2 polypeptide); (2) washing the solid substrate to remove unbound substance; and (3) eluting the ADNP polypeptide from the solid substrate, thereby obtaining the ADNP polypeptide.

The present invention also provides a composition that comprises an ADNP polypeptide and a dairy product. This ADNP polypeptide may be an ADNP1 polypeptide, which may be: (a) a full length ADNP polypeptide (SEQ ID NO:1); (b) a fragment of the full length ADNP polypeptide comprising an active core site of PGVLL (SEQ ID NO:6), ARKS (SEQ ID NO:7), SDIASHFSNKRKKCVR (SEQ ID NO:8), or K R T Y E Q M E F P L L K KRKLD (SEQ ID NO:9); or (c) a polypeptide comprising the full length ADNP polypeptide of (a) or the fragment of (b) and having up to about 40 or 20 amino acids at either or both of the N-terminus and the C-terminus. In some cases, the fragment in (b) comprises an active core site of E A R K S F L T K Y F N K Q P Y P T R R E I E K L A A S L W L W K S D I A S H F S N K R K K C V R D C E K Y K P G V L L G F N (SEQ ID NO:2).

The present invention similarly provides a composition comprising an ADNP2 polypeptide and a dairy product, and the ADNP2 polypeptide may be: (a) a full length ADNP2 polypeptide (SEQ ID NO:3); (b) a fragment of the full length ADNP2 polypeptide comprising an active core site of PSVLL (SEQ ID NO:10), EKKQ (SEQ ID NO:11), or IDVASFFGKRRYICMK (SEQ ID NO:12); or (c) a polypeptide comprising the full length ADNP2 polypeptide of (a) or the fragment of (b) and having up to about 40 amino acids at either or both of the N-terminus and the C-terminus. In some cases, the fragment of (b) comprises an active core site of E E K K Q F L K D Y F H K K P Y P S K K E I E L L S S L F W V W K I D V A S F F G K R R Y I C M K A I K N H K P S V L L G F D (SEQ ID NO:4).

In another aspect, the present invention provides a method for treating anemia using an ADNF polypeptide. The method comprises the step of administering a therapeutically effective amount of an ADNF polypeptide to a subject in need thereof. The ADNF polypeptide is a member selected from the group consisting of: (a) an ADNF I polypeptide comprising an active core site having the amino acid sequence of Ser-Ala-Leu-Leu-Arg-Ser-Ile-Pro-Ala (SEQ ID NO:13); (b) an ADNF III polypeptide comprising an active core site having the amino acid sequence of Asn-Ala-Pro-Val-Ser-Ile-Pro-Gln (SEQ ID NO:14); and (c) a mixture of the ADNF I polypeptide of part (a) and the ADNF III polypeptide of part (b). In some cases, the ADNF polypeptide is a member selected from the group consisting of a full length ADNF I polypeptide, a full length ADNF III polypeptide, and a mixture of a full length ADNF I polypeptide and a full length ADNF III polypeptide. In one example, the ADNF polypeptide is an ADNF I polypeptide. In certain embodiments, the active core site of the ADNF I polypeptide comprises at least one D-amino acid, or all D-amino acids.

In some cases, the ADNF I polypeptide has the formula (R¹)ₓ-Ser-Ala-Leu-Leu-Arg-Ser-Ile-Pro-Ala-(R²)_{y} (SEQ ID NO:15) in which: R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one.

The ADNF I polypeptide may be selected from the group consisting of: Val-Leu-Gly-Gly-Gly-Ser-Ala-Leu-Leu-Arg-Ser-Ile-Pro-Ala (SEQ ID NO:16); Val-Glu-Glu-Gly-Ile-Val-Leu-Gly-Gly-Gly-Ser-Ala-Leu-Leu-Arg-Ser-Ile-Pro-Ala (SEQ ID NO:17); Leu-Gly-Gly-Gly-Ser-Ala-Leu-Leu-Arg-Ser-Ile-Pro-Ala (SEQ ID NO:18); Gly-Gly-Gly-Ser-Ala-Leu-Leu-Arg-Ser-Ile-Pro-Ala (SEQ ID NO:19); Gly-Gly-Ser-Ala-Leu-Leu-Arg-Ser-Ile-Pro-Ala (SEQ ID NO:20); Gly-Ser-Ala-Leu-Leu-Arg-Ser-Ile-Pro-Ala (SEQ ID NO:21); and Ser-Ala-Leu-Leu-Arg-Ser-Ile-Pro-Ala (SEQ ID NO:13). In one example, the ADNF I polypeptide is Ser-Ala-Leu-Leu-Arg-Ser-Ile-Pro-Ala (SEQ ID NO:13). In some cases, the ADNF I polypeptide comprises up to about 20 amino acids at either or both of the N-terminus and the C-terminus of the active core site.

In some embodiments, the ADNF polypeptide used in the method is an ADNF III polypeptide. In other cases, the active core site of the ADNF III polypeptide comprises at least one D-amino acid, or all D-amino acids.

In some embodiments, the ADNF III polypeptide has the formula (R¹)ₓ-Asn-Ala-Pro-Val-Ser-Ile-Pro-Gln-(R²)_{y} (SEQ ID NO:22) in which: R¹ is an amino acid sequence comprising from 1 to about 40 amino acids wherein each amino acid is independently selected from the group consisting of naturally occurring amino acids and amino acid analogs; R² is an amino acid sequence comprising from 1 to about 40 amino acids wherein each amino acid is independently selected from the group consisting of naturally occurring amino acids and amino acid analogs; and x and y are independently selected and are equal to zero or one.

The ADNF III polypeptide may be a member selected from the group consisting of: Gly-Gly-Asn-Ala-Pro-Val-Ser-Ile-Pro-Gln (SEQ ID NO:23); Leu-Gly-Gly-Asn-Ala-Pro-Val-Ser-Ile-Pro-Gln-Gln-Ser (SEQ ID NO:24); Leu-Gly-Leu-Gly-Gly-Asn-Ala-Pro-Val-Ser-Ile-Pro-Gln-Gln-Ser (SEQ ID NO:25); Ser-Val-Arg-Leu-Gly-Leu-Gly-Gly-Asn-Ala-Pro-Val-Ser-Ile-Pro-Gln-Gln-Ser (SEQ ID NO:26); and Asn-Ala-Pro-Val-Ser-Ile-Pro-Gln (SEQ ID NO:14). In one example, the ADNF III polypeptide is Asn-Ala-Pro-Val-Ser-Ile-Pro-Gln (SEQ ID NO:14). In some cases, the ADNF III polypeptide comprises up to about 20 amino acids at either or both of the N-terminus and the C-terminus of the active core site.

In some embodiments of the method, a mixture of the ADNF I polypeptide of part (a) and the ADNF III polypeptide of part (b) are co-administered to the subject. In certain cases, either or both active core sites of the ADNF I polypeptide and the ADNF III polypeptide comprise at least one D-amino acid, or all D-amino acids. In the co-administration cases, the ADNF I polypeptide can be a member selected from the group consisting of: Val-Leu-Gly-Gly-Gly-Ser-Ala-Leu-Leu-Arg-Ser-Ile-Pro-Ala (SEQ ID NO:16); Val-Glu-Glu-Gly-Ile-Val-Leu-Gly-Gly-Gly-Ser-Ala-Leu-Leu-Arg-Ser-Ile-Pro-Ala (SEQ ID NO:17); Leu-Gly-Gly-Gly-Ser-Ala-Leu-Leu-Arg-Ser-Ile-Pro-Ala (SEQ ID NO:18); Gly-Gly-Gly-Ser-Ala-Leu-Leu-Arg-Ser-Ile-Pro-Ala (SEQ ID NO:19); Gly-Gly-Ser-Ala-Leu-Leu-Arg-Ser-Ile-Pro-Ala (SEQ ID NO:20); Gly-Ser-Ala-Leu-Leu-Arg-Ser-Ile-Pro-Ala (SEQ ID NO:21); and Ser-Ala-Leu-Leu-Arg-Ser-Ile-Pro-Ala (SEQ ID NO:13), whereas the ADNF III polypeptide can be selected from the group consisting of: Gly-Gly-Asn-Ala-Pro-Val-Ser-Ile-Pro-Gln (SEQ ID NO:23); Leu-Gly-Gly-Asn-Ala-Pro-Val-Ser-Ile-Pro-Gln-Gln-Ser (SEQ ID NO:24); Leu-Gly-Leu-Gly-Gly-Asn-Ala-Pro-Val-Ser-Ile-Pro-Gln-Gln-Ser (SEQ ID NO:25); Ser-Val-Arg-Leu-Gly-Leu-Gly-Gly-Asn-Ala-Pro-Val-Ser-Ile-Pro-Gln-Gln-Ser (SEQ ID NO:26); and Asn-Ala-Pro-Val-Ser-Ile-Pro-Gln (SEQ ID NO:14). In one example, the ADNF I polypeptide is Ser-Ala-Leu-Leu-Arg-Ser-Ile-Pro-Ala (SEQ ID NO:13), and wherein the ADNF III polypeptide is Asn-Ala-Pro-Val-Ser-Ile-Pro-Gln (SEQ ID NO:14).

Also in the context of co-administration of the ADNF I polypeptide and ADNF III polypeptide, the ADNF I polypeptide may comprise up to about 20 amino acids at either or both of the N-terminus and the C-terminus of the active core site of the ADNF I polypeptide, and the ADNF III polypeptide comprises up to about 20 amino acids at either or both of the N-terminus and the C-terminus of the active core site of the ADNF III polypeptide.

Typically, the ADNF polypeptide may be administered intranasally, orally, intravenously, or subcutaneously. One possible route of administration of the ADNF polypeptide is by injection, such as by intravenous injection. Another route of administration is oral ingestion of an effective amount of a dairy product. In the alternative, the ADNF polypeptide is administered with a dairy product.

In a further aspect, the present invention provides a method for isolating a full length ADNF polypeptide (an ADNF I or ADNF III polypeptide). This method comprises the step of (1) contacting, under conditions permitting antigen-antibody binding, a dairy product in an aqueous solution with a solid substrate comprising an immobilized antibody that specifically binds the ADNF polypeptide (an ADNF I or ADNF III polypeptide); (2) washing the solid substrate to remove unbound substance; and (3) eluting the ADNF polypeptide (i.e., the respectively ADNF I or ADNF III polypeptide) from the solid substrate. The isolation method therefore may be used for isolating a full length ADNF I polypeptide or a full length ADNF III polypeptide.

In a related aspect, the invention also provides a composition comprising an effective amount of an ADNF polypeptide and a dairy product, wherein the ADNF polypeptide is a member selected from the group consisting of: (a) an ADNF I polypeptide comprising an active core site having the amino acid sequence of Ser-Ala-Leu-Leu-Arg-Ser-Ile-Pro-Ala (SEQ ID NO:13); (b) an ADNF III polypeptide comprising an active core site having the amino acid sequence of Asn-Ala-Pro-Val-Ser-Ile-Pro-Gln (SEQ ID NO:14); and (c) a mixture of the ADNF I polypeptide of part (a) and the ADNF III polypeptide of part (b).

In summary, the present invention resides in the inventors' discovery of important role of ADNP and related proteins in erythropoiesis. This discovery provides a novel therapeutic and preventive approach for alleviating the symptoms of anemia as well as for reducing the risk of a patient developing anemia. Although ADNP or ADNF polypeptides have been described in the context of treating various conditions such as those involving or leading to neuronal cell death (e.g., neurodegenerative conditions, impaired learning/memory or cognitive ability, or fetal alcohol syndrome), attention deficit disorder, schizophrenia, tauopathies, retinal injury caused by laser etc., the patients to be treated by the method of this invention are typically not suffering from or at risk of developing any other conditions that may require administration of an ADNP or ADNF polypeptide. For example, the patients receiving the therapy described herein typically do not receive co-administration of an anti-cancer agent for the purpose of treating cancer or neoplasia.

In addition to the use of the ADNP or ADNF polypeptide as described in the previous sections, a nucleic acid encoding the ADNP or ADNF polypeptide described above may also be administered in an effective amount to a patient who is suffering from anemia or at risk of developing anemia for the purpose of treating anemia or reducing the risk of developing anemia. The nucleic acid may be administered in the form of pharmaceutical composition that comprises an excipient, and by the same routes of ADNP or ADNF polypeptide administration, e.g., intranasally, orally, intravenously, or subcutaneously.

### BRIEF DESCRIPTION OF THE DRAWINGS

FIG. 1: Identification and expression profile of ADNP and ADNP2 zebrafish orthologs. (A) Amino acid identity and similarity between zebrafish, mouse and human ADNP and ADNP2 (%). (B) Multiple sequence alignment (MSA) of ADNP and ADNP2 in zebrafish, mouse and human. 2 representative highly conserved regions are shown: the homeodomain (top) and one of the zinc finger domains (below, note the high conservation, especially along the 2 cysteines and 2 histidines which are the functional residues of this domain). (C) Expression profile of zebrafish adnp1 and adnp2 during embryonic development by whole-mount in situ hybridization (WMISH). 1-cell stage to 72 hours post fertilization-hpf (i-vi) are shown. (i-ii) are side views, (iii-vi) are lateral views with anterior to the top and ventral to the left. N≦15 embryos/staining.

FIG. 2: Silencing of zebrafish adnp1 and adnp2 causes impaired hematopoiesis. (A, table) Specific morpholino antisense oligodeoxynucleotides (MO's) were injected to zebrafish embryos and blood phenotypes were observed during 10 days of development. Analyses of control-MO, adnp1a-MO, adnp1b-MO and adnp2b-MO were performed at 72 hpf and analysis of adnp2a-MO was performed at 48 hpf. 4-6 independent experiments were performed for each MO. (B, table) O-dianisidine staining was performed at 48 hpf and 72 hpf to assess Hb content (N≧15 embryos/staining). (C) 48 hpf control-MO (top), adnp1a-MO (middle) and adnp1b-MO (bottom) injected embryos. Head and eyes of adnp1a and adnp1b MO's-injected embryos are smaller than those of control-MO embryo. Red blood is visible in the heart of control-MO embryo and not in adnp1a or adnp1b-MO's injected embryos. (D) 72 hpf wild type embryo (top) and adnp2a-MO injected embryo (bottom). Red blood is visible in the heart of wild-type embryo but not in adnp2a-MO injected embryo (arrows). Cardiac edema is notable in adnp2a-MO injected embryo. (E) Wild-type and adnp2a-MO injected embryos at 8 dpf. KD embryos suffer from significant edema. (F) O-dianisidine staining at 48 hpf. Reduced staining is noticeable in adnp1a, adnp1b and adnp2b-MO's injected embryos compared with control embryos. No staining is visible in adnp2a-MO injected embryos. Embryos are at lateral views, anterior to the top and ventral to the right. (G) 48 hpf o-dianisidine staining of several wild-type embryos versus adnp2a-MO injected embryos.

FIG. 3: Characterization of the hematopoietic defect in adnp2a-MO injected embryos by WMISH. (A,B) normal specification of the mesoderm layer into the hematopoietic lineage; (C,D) normal myeloid differentiation and (E) normal early erythroid development, in the presence of adnp2a-MO; (F-K) impaired maturation and differentiation of the erythroid lineage. A-K are lateral views with anterior to the right. A, B and E are 17-19 hpf embryos, C, D, F, H and I are 28 hpf embryos, G is 48 hpf embryos and J-K are 4 dpf embryos. N≧15 embryos/staining.

FIG. 4: ADNP and ADNP2 are required for mouse erythroid differentiation. (A-C) MEL cells were differentiated with 2% DMSO for 4 days and compared to non-differentiated (ND) cells incubated for the same time period. Results are means±S.E.M analyzed by student's t-test (*p<0.05, **p<0.01, ***p<0.001). (A) The picture depicts the red pellet (Hb) of differentiated (D) cells compared to the white pellet of ND cells. (B) mRNA expression levels of β-globin, band3 and c-kit in D versus ND MEL cells. 4 experiments were performed, each in duplicates. Data is presented in logarithmic scale as fold change from the expression level in the ND state. (C) Growth curves of D and ND MEL cells. Results are % of the number of cells at plating (100%). Experiments were repeated 3 times. (D+E) MEL cells were transfected with ADNP/ADNP2 shRNA/siRNA respectively, and subjected to 4 days differentiation with 2% DMSO. Real-Time PCR analysis determined ADNP, ADNP2, β-globin, band3 and c-kit expression levels. 4-5 experiments were performed, each in triplicates. Results, means±S.E.M were analyzed using student's t-test (*p<0.05, **p<0.01, ***p<0.001, relative to control cells, transfected with non-specific shRNA/siRNA sequences). (F+G) MEL cells were transfected with ADNP/ADNP2 shRNA/siRNA respectively, and viable cells were counted every 24 hrs during 4 days differentiation process with 2% DMSO. 2 separate experiments were performed in triplicates for every time point. Results are presented as % from the number of cells at plating (100%), and analyzed using student's t-test (**p<0.01, relative to number of control cells, transfected with non-specific siRNA or shRNA sequences).

FIG. 5: Impaired erythroid differentiating caused by ADNP/ADNP2 silencing is rescued by exogenous ADNP/ADNP2 mRNA. (A-B) MEL cells were treated with ADNP/ADNP2 shRNA/siRNA alone or together with ADNP/ADNP2 RNA. 4 days after transfection and induction of differentiation, RNA was extracted and mRNA levels of ADNP and ANDP2 were evaluated, to confirm efficacy of transfection. 5 experiments were performed, each in triplicates. Results are means±S.E.M and presented in logarithmic scale as fold change from the expression level in control cells (treated with non-specific siRNA/shRNA sequences). Statistical analysis was performed using one way ANOVA with post-hoc LSD (**p<0.01, ***p<0.001, relative to ADNP/ADNP2 shRNA/siRNA plus control RNA samples). (C) MEL cells were transfected with ADNP shRNA (sh68) plus control non-specific RNA or with sh68 plus ADNP RNA. Immediately after transfection, 2% DMSO was added to the cells for 4 days, when total protein was extracted and subjected to western blot analysis to confirm translation of exogenous ADNP RNA. The experiment was repeated 3 times, each in duplicates. One representing protein blot is shown. (D) Graphic depiction of the densitometric analysis of ADNP protein expression using α-tubulin as a normalizing protein. Results are means±S.E.M (**p<0.01, student's t-test). (E-F) MEL cells were treated with ADNP/ADNP2 shRNA/siRNA alone or together with ADNP/ADNP2 RNA respectively. Immediately after transfection, 2% DMSO was added to induce erythroid differentiation. On the 4^{th} day, RNA was extracted and mRNA levels of β-globin and band3 were evaluated. 5 different experiments were performed for either ADNP or ADNP2, each in triplicates. Results are means±S.E.M and presented as % from control levels. One way ANOVA was performed to compare between the groups with post-hoc LSD (*p<0.05, **p<0.01, ***p<0.001).

FIG. 6: Brg1 mRNA levels are influenced by ADNP and ADNP2 silencing in MEL cells. Brg1 mRNA levels were evaluated by Real-Time PCR in samples from the ADNP (A) and ADNP2 (B) KD experiments. Results, means±S.E.M (N=4 for ADNP and N=5 for ADNP2, each in triplicates) are presented as % from Brg1 levels in control cells (transfected with non-specific siRNA/shRNA). One way ANOVA was performed to compare between the groups with post hoc Tukey HSD (**p<0.01, *** p<0.001, in comparison to control cells).

FIG. 7: A. Varying amounts of milk protein (20-200 μg/lane) were loaded on the gel and ADNP-like immunoreactivity (˜100 KD) was detected in the milk samples. B. Further studies used 8000×g centrifugation for 1 hr of 5% skim milk (1 ml fractions). Supernatants (1 ml) were collected and pellets dissolved in 1 ml water. ADNP-like immunoreactivity was detected.

FIG. 8: Relative quantity of globin mRNA (RQ) correlates with increasing concentrations of milk.

### DEFINITIONS

The term “activity-dependent neuroprotective protein (ADNP)” refers to an activity dependent neurotrophic factor (ADNF) that has neurotrophic/neuroprotective activity as measured with in vitro cortical neuron culture assays described by, e.g., Hill et al., Brain Res. 603, 222-233 (1993); Gozes et al., Proc. Natl. Acad. Sci. USA 93, 427-432 (1996). Full length human ADNP has a predicted molecular weight of 123,562.8 Da (>1000 amino acid residues) and a theoretical pI of about 6.97. ADNP gene is localized to human chromosome 20q13.13-13.2, a region associated with cognitive function. Full-length amino acid and nucleic acid sequences of ADNP can be found in WO 98/35042, WO 00/27875, U.S. Pat. Nos. 6,613,740 and 6,649,411. The Accession number for the human sequence is NP_{—}852107. ADNP is also known as ADNF III. The term “ADNP2” refers to a homolog of ADNP assigned to human chromosome 18q23, a region associated with psychiatric disorder. See, e.g., Zamostiano et al., J. Biol. Chem. 276(1):708 (2001); Kushnir et al., J Neurochem, 105: 537-545 (2008); Van Broeckhoven, C. and Verheyen, G. Am J Med Genet, 88: 263-270 (1999). In this application, the term “ADNP1” is used interchangeably with “ADNP,” in some cases to more clearly distinguish from ADNP2.

As used in this application, the term “ADNP polypeptide” encompasses not only the full length ADNP and ADNP2 polypeptides but also their fragments comprising one or more of the core active sites such as those shown in SEQ ID NOs:2, 4, and 6-12. Furthermore, an ADNP polypeptide can also be a full length ADNP or ADNP2 polypeptide or a fragment thereof comprising at least one of the active core sites such as SEQ ID NOs:2, 4, and 6-12 plus some additional amino acids at either or both of the N-terminus and the C-terminus. For example, a fragment of an ADNP polypeptide may exhibit at least 90% or 95% sequence identity to SEQ ID NO:2 and also comprises the core sites of SEQ ID NOs:6-8 or the core sites of SEQ ID NOs:6-9. Similarly, a fragment of an ADNP2 polypeptide may exhibit at least 90% or 95% sequence identity to SEQ ID NO:4 and also comprise the core sites of SEQ ID NOs:10-12 or the core sequences of SEQ ID NOs:10-12 plus SEQ ID NO:9. SEQ ID NO:9 is the nuclear localization signal in ADNP polypeptides. An ADNP or ADNP2 polypeptide useful in this invention possesses the activity of promoting erythropoiesis, which can be tested and verified in the murine erythroleukemia cell line (MEL) globin production assays described in Example 3.

The phrase “ADNF polypeptide” refers to one or more activity-dependent neurotrophic factors (ADNF) that have an active core site comprising the amino acid sequence of SALLRSIPA (referred to as “SAL”) or NAPVSIPQ (referred to as “NAP”), or conservatively modified variants thereof that have neurotrophic/neuroprotective activity as measured with in vitro cortical neuron culture assays described by, e.g., Hill et al., Brain Res. 603:222-233 (1993); Brenneman & Gozes, J. Clin. Invest. 97:2299-2307 (1996), Gozes et al., Proc. Natl. Acad. Sci. USA 93, 427-432 (1996). An ADNF polypeptide can be an ADNF I polypeptide, an ADNF III polypeptide, their alleles, polymorphic variants, analogs, interspecies homolog, any subsequences thereof (e.g., SALLRSIPA or NAPVSIPQ) or lipophilic variants that exhibit neuroprotective/neurotrophic action on, e.g., neurons originating in the central nervous system either in vitro or in vivo. An “ADNF polypeptide” can also refer to a mixture of an ADNF I polypeptide and an ADNF III polypeptide.

The terms “ADNF I polypeptide” and “ADNF I” refer to an activity dependent neurotrophic factor polypeptide having a molecular weight of about 14,000 Daltons with a pI of 8.3±0.25. As described above, ADNF I polypeptides have an active site comprising an amino acid sequence of Ser-Ala-Leu-Leu-Arg-Ser-Ile-Pro-Ala (also referred to as “SALLRSIPA” or “SAL” or “ADNF-9”). See Brenneman & Gozes, J. Clin. Invest. 97:2299-2307 (1996), Glazner et al., Anat. Embryol. 200:65-71 (1999), Brenneman et al., J. Pharm. Exp. Ther., 285:619-27 (1998), Gozes & Brenneman, J. Mol. Neurosci. 7:235-244 (1996), and Gozes et al., Dev. Brain Res. 99:167-175 (1997). Unless indicated as otherwise, “SAL” refers to a peptide having an amino acid sequence of Ser-Ala-Leu-Leu-Arg-Ser-Ile-Pro-Ala, not a peptide having an amino acid sequence of Ser-Ala-Leu. Some of the active fragments of ADNF I are described in International PCT Publication No. WO 96/11948.

The terms “ADNF III polypeptide” and “ADNF III,” also known as activity-dependent neuroprotective protein (ADNP), refer to one or more activity dependent neurotrophic factors (ADNF) that have an active core site comprising the amino acid sequence of NAPVSIPQ (referred to as “NAP”), or conservatively modified variants thereof that have neurotrophic/neuroprotective activity as measured with in vitro cortical neuron culture assays described by, e.g., Hill et al., Brain Res. 603, 222-233 (1993); Gozes et al., Proc. Natl. Acad. Sci. USA 93, 427-432 (1996). An ADNF polypeptide can be an ADNF III polypeptide, allelic or polymorphic variant, analog, interspecies homolog, or any subsequences thereof (e.g., NAPVSIPQ) that exhibit neuroprotective/neurotrophic action on, e.g., neurons originating in the central nervous system either in vitro or in vivo. ADNF III polypeptides can range from about eight amino acids and can have, e.g., between 8-20, 8-50, 10-100 or about 1000 or more amino acids.

Full length human ADNF III has a predicted molecular weight of 123,562.8 Da (>1000 amino acid residues) and a pI of about 6.97. As described above, ADNF III polypeptides have an active site comprising an amino acid sequence of Asn-Ala-Pro-Val-Ser-Ile-Pro-Gln (also referred to as “NAPVSIPQ” or “NAP”). See Zamostiano et al., J. Biol. Chem. 276:708-714 (2001) and Bassan et al., J. Neurochem. 72:1283-1293 (1999). Unless indicated as otherwise, “NAP” refers to a peptide having an amino acid sequence of Asn-Ala-Pro-Val-Ser-Ile-Pro-Gln, not a peptide having an amino acid sequence of Asn-Ala-Pro. Full-length amino acid and nucleic acid sequences of ADNF III can be found in International PCT Publication Nos. WO 98/35042 and WO 00/27875, and U.S. Pat. No. 6,613,740. The Accession number for the human sequence is NP_{—}852107, see also Zamostiano et al., supra.

Similar to the ADNP polypeptides, the ADNF I and ADNF III polypeptides useful for the present invention possess the activity of promoting hematopoiesis, which can be tested and verified by the MEL globin production assays described in Example 3.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. For the purposes of this application, amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. For the purposes of this application, amino acid mimetics refers to chemical compounds that have a structure that is different from the general chemical structure of an amino acid, but that functions in a manner similar to a naturally occurring amino acid.

Amino acids may include those having non-naturally occurring D-chirality, as disclosed in International PCT Publication No. WO 01/12654, incorporated herein by reference, which may improve oral availability and other drug like characteristics of the compound. In such embodiments, one or more, and potentially all of the amino acids of NAP or the ADNF polypeptide will have D-chirality. The therapeutic use of peptides can be enhanced by using D-amino acids to provide longer half life and duration of action. However, many receptors exhibit a strong preference for L-amino acids, but examples of D-peptides have been reported that have equivalent activity to the naturally occurring L-peptides, for example, pore-forming antibiotic peptides, beta amyloid peptide (no change in toxicity), and endogenous ligands for the CXCR4 receptor. In this regard, NAP and ADNF polypeptides also retain activity in the D-amino acid form (Brenneman et al., J. Pharmacol. Exp. Ther. 309(3):1190-7 (2004)).

Amino acids may be referred to by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following groups each contain amino acids that are conservative substitutions for one another:

1) Alanine (A), Glycine (G);

2) Serine (S), Threonine (T);

3) Aspartic acid (D), Glutamic acid (E);

4) Asparagine (N), Glutamine (Q);

5) Cysteine (C), Methionine (M);

6) Arginine (R), Lysine (K), Histidine (H);

7) Isoleucine (1), Leucine (L), Valine (V); and

8) Phenylalanine (F), Tyrosine (Y), Tryptophan (W). (see, e.g., Creighton, Proteins (1984)).

The terms “polypeptide,” “peptide” and “protein” are used interchangeably herein to refer to a polymer of amino acid residues. Generally, a peptide refers to a short polypeptide. The terms apply to amino acid polymers in which one or more amino acid residue is an analog or mimetic of a corresponding naturally occurring amino acid, as well as to naturally occurring amino acid polymers.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains.

The term “antibody” also refers to antigen- and epitope-binding fragments of antibodies, e.g., Fab fragments, that can be used in immunological affinity assays. There are a number of well characterized antibody fragments. Thus, for example, pepsin digests an antibody C-terminal to the disulfide linkages in the hinge region to produce F(ab)′₂, a dimer of Fab which itself is a light chain joined to VH-CH1 by a disulfide bond. The F(ab)′₂ can be reduced under mild conditions to break the disulfide linkage in the hinge region thereby converting the (Fab′)₂ dimer into an Fab′ monomer. The Fab′ monomer is essentially a Fab with part of the hinge region (see, e.g., Fundamental Immunology, Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody also includes antibody fragments either produced by the modification of whole antibodies or synthesized using recombinant DNA methodologies.

The phrase “specifically binds,” when referring to a protein or peptide, refers to a binding reaction that is determinative of the presence of the protein in a heterogeneous population of proteins and other biologics. Thus, under designated immunoassay conditions, the specified binding agent (e.g., an antibody) binds to a particular protein at least two times the background and does not substantially bind in a significant amount to other proteins present in the sample. Specific binding to an antibody under such conditions may require an antibody that is selected for its specificity for a particular protein or a protein but not its similar “sister” proteins. For example, antibodies may be raised to specifically bind ADNP protein but not ADNP2 protein. In the alternative, antibodies can be raised and selected to specifically bind ADNP2 protein but not ADNP protein. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background.

The term “subject” refers to any mammal, in particular human, at any stage of life.

The term “contacting” is used herein interchangeably with the following: combined with, added to, mixed with, passed over, incubated with, flowed over, etc. Moreover, the ADNP or ADNF polypeptides of the present invention can be “administered” by any conventional method such as, for example, parenteral (e.g., intravenous, subcutaneous, intradermally or intramuscularly), oral, topical, intravitreal and inhalation (e.g., intranasal) routes.

As used herein “treatment” includes both therapeutic and preventative treatment of a condition, such as treatment for alleviating ongoing symptoms and prevention of disease progression or onset of further symptoms, or for avoidance or reduction of side-effects or symptoms of a disease. As used herein the term “prevent” and its variations do not require 100% elimination of the occurrence of an event; rather, the term and its variation refer to an inhibition or reduction in the likelihood of such occurrence.

As used herein, “condition” and “disease” include incipient conditions or disorders, or symptoms of a disease, incipient condition or disorder.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that protein gives rise to essentially one band in an electrophoretic gel. Particularly, it means that the protein is at least 85% pure, more preferably at least 95% pure, and more preferably at least 99% pure.

“An amount sufficient” or “an effective amount” or a “therapeutically effective amount” is that amount of a therapeutic agent at which the agent exhibits its activity for the intended purpose of its administration. In therapeutic applications, an amount adequate to accomplish this is defined as the “therapeutically effective dose.” For example, an effective amount for an ADNP or ADNF polypeptide of the invention is an amount that when administered to a patient suffering from anemia or at risk of developing anemia, the polypeptide is capable to reducing or substantially eliminating the symptoms of the condition or reducing or substantially eliminating the risk of developing the condition. An effective amount of a dairy product, when administered alone for the purpose of treating or preventing anemia, is similarly defined as an amount sufficient to reduce or substantially eliminate the symptoms of anemia or to reduce or substantially eliminate the risk of developing the condition. An effective amount of a dairy product, when administered together with an ADNP or ADNF polypeptide of this invention for the purpose of treating or preventing anemia, is defined as an amount sufficient to show any discernable augmentation in the effect of ADNP or ADNF polypeptide, to reduce or substantially eliminate the symptoms of anemia or to reduce or substantially eliminate the risk of developing the condition. An effective amount may vary with the particular ADNP or ADNF polypeptide used, the route of administration, and the particular formulation of a composition, including a dairy product.

The term “anemia” is used in this application to refer to a blood disorder that involves decreased oxygen-carrying ability of hemoglobin (found in red blood cells) in a patient, which may be caused by impaired production of red blood cells, abnormally high rate of destruction or loss of red blood cells, or abnormal shape and/or size of red blood cells. The clinical criteria for diagnosing anemia are well known to the average skilled medical professionals. For example, anemia is typically defined as hemoglobin level of less than 13.5 gram/100 ml in men and less than 12.0 gram/100 ml in women, although these standards may vary slightly depending on the source and the laboratory reference used.

As used herein, the term “dairy product” encompasses the milk of any mammal and any product made from modifying the milk by altering its water and/or fat contents and/or adding one or more ingredients not naturally present in the milk. For example, dried milk powder, reduced fat milk, skim milk, and vitamin fortified milk are encompassed by the term “dairy product” in this application.

### DETAILED DESCRIPTION OF THE INVENTION

### I. ADNP and ADNF Polypeptides

The ADNP polypeptides and ADNF polypeptides useful in this invention can be obtained by chemical synthesis, recombinant production, or, in the case of full length ADNP or ADNF polypeptides, isolation from a source where the proteins are naturally present.

### A. Chemical Synthesis

The Polypeptides useful according to this invention can be produced chemically, e.g., by systematically adding one amino acid at a time, followed by screening of the resulting peptide for biological activity, as described herein. In some cases, one or more of the amino acids in the core active sites may be substituted by a D-amino acid. In addition, various substitutions may be made to amino acid residues outside of the core sites.

Polypeptides comprising non-standard amino acids can also be made. In some embodiments, at least one of the amino acids of the active core sequence is a non-standard amino acid. In some embodiments, 2, 3, 4, 5, or more of the amino acids is a non-standard amino acid. In some cases, all amino acids are non-standard amino acid (such as D-amino acid) in a core active site. Examples of non-standard amino acids are alpha-aminoisobutyric acid, N-methylated amino acids, amino acids with a D chiral center, aza-tryptophan, etc. A wide range of non-standard amino acids are commercially available, e.g., at Genzyme Pharmaceuticals (Cambridge, Mass.).

Polypeptide sequences, including those with non-standard amino acids, can be generated synthetically using commercially available peptide synthesizers to produce any desired polypeptide (see Merrifield, Am. Chem. Soc. 85:2149-2154 (1963); Stewart & Young, Solid Phase Peptide Synthesis (2nd ed. 1984)). Various automatic synthesizers and sequencers are commercially available and can be used in accordance with known protocols (see, e.g., Stewart & Young, Solid Phase Peptide Synthesis (2nd ed. 1984)). Solid phase synthesis in which the C-terminal amino acid of the sequence is attached to an insoluble support followed by sequential addition of the remaining amino acids, or non-standard amino acids, in the sequence is a method for the chemical synthesis of the peptides of this invention. Techniques for solid phase synthesis are described by Barany & Merrifield, Solid-Phase Peptide Synthesis; pp. 3-284 in The Peptides: Analysis, Synthesis, Biology. Vol. 2: Special Methods in Peptide Synthesis, Part A.; Merrifield et al 1963; Stewart et al. 1984). NAP and NAP-IBA peptides can be synthesized using standard Fmoc protocols (Wellings & Atherton, Methods Enzymol. 289:44-67 (1997)).

### B. Recombinant Production

In addition to chemical synthesis, the polypeptides for use in the invention can be prepared by recombinant DNA methodology. Generally, this involves creating a nucleic acid sequence that encodes the polypeptide, placing the nucleic acid in an expression cassette under the control of a particular promoter, and expressing the protein in a host cell. Recombinantly engineered cells known to those of skill in the art include, but are not limited to, bacteria, yeast, plant, filamentous fungi, insect (especially employing baculoviral vectors), and mammalian cells.

The recombinant nucleic acids are operably linked to appropriate control sequences for expression in the selected host. For E. coli, exemplary control sequences include the T7, trp, or lambda promoters, a ribosome binding site and, optionally, a transcription termination signal. For eukaryotic cells, the control sequences can include a promoter and, optionally, an enhancer, e.g., derived from immunoglobulin genes, SV40, cytomegalovirus, etc., a polyadenylation sequence, and splice donor and acceptor sequences.

The plasmids of the invention can be transferred into the chosen host cell by methods such as, for example, the calcium chloride transformation method for E. coli and the calcium phosphate treatment or electroporation methods for mammalian cells. Cells transformed by the plasmids can be selected by resistance to antibiotics conferred by genes contained on the plasmids, such as the amp, gpt, neo, and hyg genes.

Once expressed, the recombinant peptides can be purified according to standard procedures of the art, including ammonium sulfate precipitation, affinity columns, column chromatography, gel electrophoresis and the like (see, e.g., Scopes, Polypeptide Purification (1982); Deutscher, Methods in Enzymology Vol. 182: Guide to Polypeptide Purification (1990)). Optional additional steps include isolating the expressed protein to a higher degree, and, if required, cleaving or otherwise modifying the peptide, including optionally renaturing the protein.

One of skill can select a desired nucleic acid or polypeptide of the invention based upon the sequences provided and upon knowledge in the art regarding proteins generally. Knowledge regarding the nature of proteins and nucleic acids allows one of skill to select appropriate sequences with activity similar or equivalent to the nucleic acids and polypeptides disclosed herein.

One of skill will recognize many ways of generating alterations in a nucleic acid sequence encoding a given peptide sequence. Polypeptide sequences can also be altered by changing the corresponding nucleic acid sequence and expressing the polypeptide. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other known techniques (see Giliman & Smith, Gene 8:81-97 (1979); Roberts et al., Nature 328:731-734 (1987)).

After chemical synthesis, biological expression or purification, the polypeptide(s) may possess a conformation substantially different than the native conformations of the constituent polypeptides. In this case, it is helpful to denature and reduce the polypeptide and then to cause the polypeptide to re-fold into the preferred conformation. Methods of reducing and denaturing peptides and inducing re-folding are known to those of skill in the art (see Debinski et al., J. Biol. Chem. 268:14065-14070 (1993); Kreitman & Pastan, Bioconjug. Chem. 4:581-585 (1993); and Buchner et al., Anal. Biochem. 205:263-270 (1992)). Debinski et al., for example, describe the denaturation and reduction of inclusion body peptides in guanidine-DTE. The peptide is then refolded in a redox buffer containing oxidized glutathione and L-arginine

The ADNP and ADNF polypeptides used in this invention can be evaluated by screening techniques in suitable assays for the desired characteristic, i.e., promoting hematopoiesis. For instance, changes in the immunological character of a polypeptide can be detected by an appropriate immunological assay. Modifications of other properties such as nucleic acid hybridization to a target nucleic acid, redox or thermal stability of a protein, hydrophobicity, susceptibility to proteolysis, or the tendency to aggregate can be assayed. More particularly, the small peptides of the present invention can be screened by employing suitable assays and animal models known to those skilled in the art.

One of skill will recognize that modifications can be made to the polypeptides without diminishing their biological activity. Some modifications may be made to facilitate the cloning, expression, or intake of the polypeptide by the target cells or tissue. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

### C. Isolation of ADNP and ADNP2 Polypeptides

It is also possible to obtain the full length ADNP and ADNF polypeptides by isolating them from their natural sources. The present inventors in their studies recognized that full length ADNP and ADNP2 proteins are present in the milk of mammalian species. This revelation not only provides an important source for one to obtain the full length ADNP and ADNP2 polypeptides but also allows the use of dairy products in the context of treating patients suffering from anemia.

The isolation of a full length ADNP or ADNF polypeptide may be achieved by a variety of methods known in the field of protein isolation. For example, the polypeptide may be isolated based on its chemical and physical characteristics such as molecular weight, iso-electrical point, and specific binding affinity. Various chromatographic methods can be used to isolate the polypeptide from other components found in the milk. Different types of electrophoretic methods may also be used for isolation purpose. In some cases, an affinity-based methodology may be a preferred approach to purify a target polypeptide, such as an ADNP or ADNF polypeptide, from a dairy product. For example, an antibody that specifically binds the target polypeptide can be immobilized on a solid substrate (e.g., a column, plate, or spherical particle such as a bead). When a dairy product in an aqueous solution is incubated with the antibody-coated substrate under conditions that favor antigen-antibody binding, the target polypeptide will bind to the immobilized antibody and therefore retain on the substrate. Once the unbound substance from the dairy product is washed off from the solid substrate, one may then elude the target polypeptide from the substrate under changed conditions (e.g., changed pH and/or ionic strength) where antigen-antibody binding tends to be disrupted and obtain a solution that contains purified target polypeptide.

### D. Functional Assays of the Polypeptides

The ADNP or ADNF polypeptides as described herein are useful for the method of this invention due to their activity in promoting erythropoiesis. After its production (synthetically or recombinantly) or purification, an ADNP or ADNF polypeptide, especially one that comprises only less than full length of SEQ ID NO:1, 3, or 5, or one that comprises substituted amino acids (including substitution with alternative amino acids or with non-naturally occurring amino acids such as D-amino acids), must therefore be tested for its erythropoiesis-promoting activity before its use in the claimed method. Methods are known in the art for this purpose and one example, the MEL assay, is provided in detail in this application in Example 3.

### II. Pharmaceutical Compositions and Administration

The pharmaceutical compositions comprising the ADNP or ADNF polypeptide of this invention are suitable for use in a variety of drug delivery systems. The polypeptides can be administered systemically, e.g., by injection (intravenous, subcutaneous, intradermal, or intramuscular), or by orally administration, or by nasal administration, or a local administration such as using a dermal patch etc. The methods for various routes of delivery are well known to those of skill in the art.

Suitable formulations for use in the present invention are found in Remington's Pharmaceutical Sciences (17th ed. 1985)). For a brief review of methods for drug delivery, see Langer, Science 249:1527-1533 (1990). Suitable dose ranges are described in the examples provided herein, as well as in WO96/11948.

As such, the present invention provides for therapeutic compositions or medicaments comprising one or more of the polypeptides described hereinabove in combination with a pharmaceutically or physiologically acceptable excipient, wherein the amount of polypeptide is sufficient to provide a therapeutic effect, e.g., to improve the anemic condition a patient is receiving the treatment for.

In a therapeutic application, the polypeptides of the present invention are embodied in pharmaceutical compositions intended for administration by any effective means, including parenteral, topical, oral, nasal, pulmonary (e.g. by inhalation) or local administration. Nasal pumps, eye drops, and topical patches can be used.

The invention provides compositions for parenteral administration that comprise a solution of polypeptide, as described above, dissolved or suspended in an acceptable carrier, such as an aqueous carrier. Parenteral administration can comprise, e.g., intravenous, subcutaneous, intradermal, intramuscular, or intranasal administration. A variety of aqueous carriers may be used including, for example, water, buffered water, 0.4% saline, 0.3% glycine, hyaluronic acid and the like. These compositions may be sterilized by conventional, well known sterilization techniques or, they may be sterile filtered. The resulting aqueous solutions may be packaged for use as is or lyophilized, the lyophilized preparation being combined with a sterile solution prior to administration. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride potassium chloride, calcium chloride, sorbitan monolaurate, triethanolamine oleate, etc.

For solid compositions, conventional nontoxic solid carriers may be used that include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient and more preferably at a concentration of 25%-75%.

For aerosol administration, the polypeptides are preferably supplied in finely divided form along with a surfactant and propellant. Accordingly, in some embodiments, the pharmaceutical composition comprises a surfactant such as a lipophilic moiety to improve penetration or activity. Lipophilic moieties are known in the art and described, e.g., in U.S. Pat. No. 5,998,368. The surfactant must be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. A carrier can also be included, as desired, as with, e.g., lecithin for intranasal delivery. An example includes a solution in which each milliliter included 7.5 mg NaCl, 1.7 mg citric acid monohydrate, 3 mg disodium phosphate dihydrate and 0.2 mg benzalkonium chloride solution (50%) (see, e.g., Gozes et al., J Mol. Neurosci. 19:167-70 (2002)).

In therapeutic applications, the polypeptides of the invention are administered to a patient in an amount sufficient to reduce or eliminate symptoms of anemia or red blood cell deficits. An amount adequate to accomplish this is defined as “therapeutically effective dose.” Amounts effective for this use will depend on, for example, the particular polypeptide employed, the particular form of a pharmaceutical composition in which the polypeptide is present (e.g., a highly purified ADNP polypeptide v. a dairy product containing ADNP polypeptides), the type of disease or disorder to be treated and its severity, the manner of administration, the weight and general state of health of the patient, and the judgment of the prescribing physician.

For example, an amount of an ADNP or ADNF polypeptide falling within the range of a 100 ng to 10 mg dose given intranasally once a day would be a therapeutically effective amount. Alternatively, dosages may be outside of this range when on a different schedule (such as by injection or oral ingestion). For example, dosages can range from 0.0001 mg/kg to 10,000 mg/kg, and can be about 0.001 mg/kg, 0.1 mg/kg, 1 mg/kg, 5 mg/kg, 50 mg/kg or 500 mg/kg per dose. Doses may be administered hourly, every 4, 6 or 12 hours, with meals, daily, every 2, 3, 4, 5, 6, or 7 days, weekly, every 2, 3, 4 weeks, monthly or every 2, 3 or 4 months, or any combination thereof. The duration of dosing may be single (acute) dosing, or over the course of days, weeks, months, or years, depending on the condition to be treated. Those skilled in the art can determine the suitable dosage and administration frequency depending on the particular circumstances of individual patients.

In some cases, the composition used for practicing the present invention is a composition that is specifically formulated for oral administration and comprises an effective amount of an ADNP or ADNF polypeptide and a dairy product. The presence of naturally occurring ADNP or ADNF polypeptides in the milk serves to enhance the therapeutic effects of the composition. On the other hand, the milk micelles act as a carrier to maintain stability of the ADNP or ADNF polypeptides and to allow easy absorption of the polypeptides. Examples of such a composition include a fortified milk drink, which is immediately consumable by anemic patients or those at risk of developing an anemic condition, or a dried mild power, which may be added to other food items or beverages for ingestion.

### EXAMPLES

The following examples are provided by way of illustration only and not by way of limitation. Those of skill in the art will readily recognize a variety of non-critical parameters that could be changed or modified to yield essentially the same or similar results.

### Example 1

### Evolutionary-Conserved Role of the ADNP Family Proteins in Erythropoiesis

### Materials and Methods

### Zebrafish Maintenance and Breeding

Maintenance and breeding were performed as before (Ziv et al., 2005, J Neuroendocrinol 17, 314-320). All experiments were performed by crossbreeding of standard wild-type strains (TL and AB).

### Isolation of Zebrafish ADNP and ADNP2 Genes

Based on alignment of human ADNP and ADNP2 sequences to the zebrafish genome, 4 ADNP and ADNP2 orthologs were identified (adnp1a and adnp1b are the 2 orthologs of the mammalian ADNP and adnp2a and adnp2b are the 2 orthologs of the mammalian ADNP2). The EMBOSS Pairwise Alignment Algorithms (Blosum62) and ClustalW were used for 2 sequence alignment or multiple sequence alignment (MSA), respectively. The coding sequence of the 4 zebrafish genes was isolated from cDNA of 24 hpf embryos. The adnp1 and adnp2 orthologs were cloned into the pGEM-T-easy vector (Promega, Madison, Wis., USA) in 2 separate fragments. These plasmids were used as a template to produce DIG-labeled anti-sense RNA for WMISH experiments.

### Morpholino Knockdown Experiments

Gene knockdown experiments were performed using morpholino-modified antisense oligonucleotides (MO's, Gene Tools, Philomath, Oreg., USA). MO's were injected into wild type embryos, immediately after fertilization (2 nl, 1-2 mM). This was followed by a daily monitoring of the embryos for 10 days. MO's sequences were chosen through the Gene Tools oligonucleotide design service. A splicing MO, directed to intron3-exon4 boundary, was designed to block adnp1a (adnp1a-MO, 5′-TAGTCCTGCAACATTTGAGAAACCA-3′). The efficiency of the adnp1a splicing MO was evaluated by RT-PCR. Translation MO's were designed to block adnp1b, adnp2a and adnp2b (adnp1 b-MO, 5′-TTGTTCACTGGGAGTTGAAACATTC-3′; adnp2a-MO: 5′-CACTGGAATCTGATACATTTTGCTC-3′; adnp2b-MO: 5′-ACCTTTTACTGGAAACTGGTACATG-3′). BLAST analysis was performed to verify that the MO's were not predicted to bind gene sequences other than their targets, and that their target sequence was not conserved among the other zebrafish adnp1 and adnp2 genes. Gene-Tools standard control-MO, which has not been reported to have other targets or generate any phenotypes, was used as a negative control (5-CCTCTTACCTCAGTTACAATTTATA-3′). Injections were repeated in at least 4 independent experiments for each MO.

### Criteria for Evaluating the Status of Zebrafish Blood

For adnp2a-MO injected embryos, blood circulation was evaluated at 48 hpf. For the 3 other MO's, circulation was examined at 72 hpf when red blood is easily seen. Based on observation, embryos were divided to have normal blood cells, no blood cells or hypochromic and/or fewer blood cells.

### O-dianisidine Staining

Hb activity was detected in whole embryos by performing o-dianisidine staining as described (Iuchi and Yamamoto, 1983, J Exp Zool 226, 409-417).

### Whole-Mount in Situ Hybridization (WMISH)

Transcripts of sci (NM_{—}213237), lmo2 (NM_{—}131111), gata1 (NM_{—}131234), band3 (NM_{—}198338), embryonic α-globin 1 (NM_{—}182940), lys-C(NM_{—}139180), l-plastin (NM_{—}131320), ikaros (NM_{—}130986), c-myb (NM_{—}131266), irx7 (NM_{—}131881), flk1 (NM_{—}131472), cmlc2 (NM_{—}131329), adnp1a (NM_{—}001080015), adnp1b (XM_{—}002666474), adnp2a (NM_{—}001098265) and adnp2b (NM_{—}001080013) were detected by WMISH using digoxygenin (DIG) labeled antisense RNA probes at a concentration of 1 ng/μl. Transcription of RNA probes was performed from pGEM-T Easy plasmids linearized before the coding sequence, using SP6 or T7 RNA polymerases (DIG RNA labeling kit, Roche Applied Science, Penzberg, Germany). Embryos at different developmental stages were fixed overnight in 4% paraformaldehyde and stored in 100% methanol. WMISH was performed as describe (Ziv et al., 2005, supra). Fixed embryos were then placed in 70% glycerol for observation and photography using a dissecting stereoscope (SZX12, Olympus) equipped with digital camera (DP70, Olympus). In each experiment, embryos hybridized with the same probe were stained under the same conditions and for the same time period using identical microscopic and camera settings.

### Murine Erythroleukemia Cell Line

MEL cells were a gift from Prof. N. Shaklai (Tel Aviv University, Israel). Cells were cultured at 37° C. in 5% CO₂ incubator in RPMI-1640 medium supplemented with 10% fetal calf serum, 2 mM L-glutamine, 100 U/ml penicillin, 0.1 mg/ml streptomycin and 12.5 U/ml nystatin (Biological Industries, Beit Haemek, Israel). To induce erythroid differentiation, 2% DMSO (v/v, Sigma, St. Louis, Mo., USA) was added for 4 days.

### RNA Interference and Transfections

2 ADNP shRNA clones (sh68 and sh71, Sigma) were used for mouse ADNP (NM_{—}009628) silencing in MEL cells, as before (Mandel et al., 2008, J Mol Neurosci 35, 127-141). To control for off-target effects, a control shRNA vector containing nonspecific green fluorescent protein fragment in pRetroSuper plasmid that does not have a match in the mouse genome was used (Mandel and Gozes, 2007, J Biol Chem 282, 34448-34456). For mouse ADNP2 (NM_{—}175028) silencing, a mixture of 2 stealth small interfering RNA (siRNA) duplexes (Invitrogen, Carlsbad, Calif., USA) was used.

Transfections were performed using Lipofectamine-2000 (Invitrogen). 6 hours after transfection, 2% DMSO was added to the cells for 4 days in order to induce erythroid differentiation. On the 4th day, cells were harvested, RNA was extracted and expression levels of ADNP, ADNP2, band3, β-globin, Brg1 and c-kit were evaluated.

### Rescue Experiments in Murine Erythroleukemia Cells

Mouse ADNP and ADNP2 RNA were synthesized using the mMES SAGE mMACHINE kit with T7 promoter (Ambion, Austin, Tex., USA) from linearized pGEM-T Easy plasmids containing the coding sequence of mouse ADNP and ADNP2. Control RNA encoding elongation factor 1α in Xenopus was synthesized in order to monitor for non-specific effects.

Cells were transfected (Lipofectamine 2000) using 5 experimental conditions: (1) control non-specific shRNA/siRNA (2) Specific ADNP/ADNP2 shRNA/siRNA (3) ADNP/ADNP2 shRNA/siRNA plus control RNA (4) ADNP/ADNP2 shRNA/siRNA plus ADNP/ADNP2 RNA (5) Control non-specific shRNA/siRNA plus ADNP/ADNP2 RNA.

4 hours after transfection, differentiation was initiated by adding 500 μl of normal growth medium supplemented with 2% DMSO. The differentiation process continued until the 4th day, when RNA was extracted and Real-Time PCR analysis was carried out to evaluate ADNP, ADNP2, band3, and β-globin mRNA expression.

### RNA Extraction

Total RNA from MEL cells was extracted by the RNeasy Plus Mini Kit (Qiagen, Hilden, Germany). RNA integrity was determined by fractionation on 1% agarose gel and staining with ethidium bromide. RNA quantity (OD260) was determined by NanoDrop spectrophotometer (NanoDrop Technologies, Wilmington, Del., USA).

### Reverse Transcription and Quantitative Real Time PCR

Samples with the same amount of total RNA were used to synthesize single-strand cDNA employing the Applied Biosystems High Capacity cDNA Reverse Transcription Kit (Applied Biosystems, Foster City, Calif., USA) and random hexamers primers as before (Dresner et al., 2010, supra). Primer pairs (see Table 1) were designed using the primer 3 web interface (website: frodo.wi.mit.edu/primer3/) and synthesized by Sigma-Genosys (The Woodlands, Tex., USA). To avoid amplification of contaminating genomic DNA, primers were designed to exon-exon junction. Real Time PCR was performed using the SYBR® Green PCR Master Mix and ABI PRISM 7900 Sequence Detection System instrument and software (Applied Biosystems) as previously described (Dresner et al., 2010, supra). 18S ribosomal RNA was used as the normalization control gene. Product specificity was confirmed in the initial experiments by agarose gel electrophoresis and sequencing and routinely by melting curve analysis.

### Protein Extraction and Western Blot Analysis

Total protein extraction from MEL cells was performed using RIPA buffer (50 mM Tris pH 7.5, 150 mM NaCl, 1% NP-40, 0.1% SDS and 0.5% deoxycholic acid) supplemented with protease inhibitors cocktail (Sigma) and 5 mM EDTA. Proteins were quantified and subjected to western blot analysis using mouse monoclonal anti-ADNP (1:400, BD Biosciences, San Jose, Calif., USA) and mouse monoclonal anti-α-tubulin (1:1000, clone DM1A, Sigma) as an internal standard.

### Statistical Analyses

Student's t-test for the comparisons of 2 variables and ANOVA for the comparisons of multiple variables with the appropriate post hoc analyses were used as indicated in the figure legends.

### Results

### Identification and Expression Profile of ADNP and ADNP2 Zebrafish Orthologs

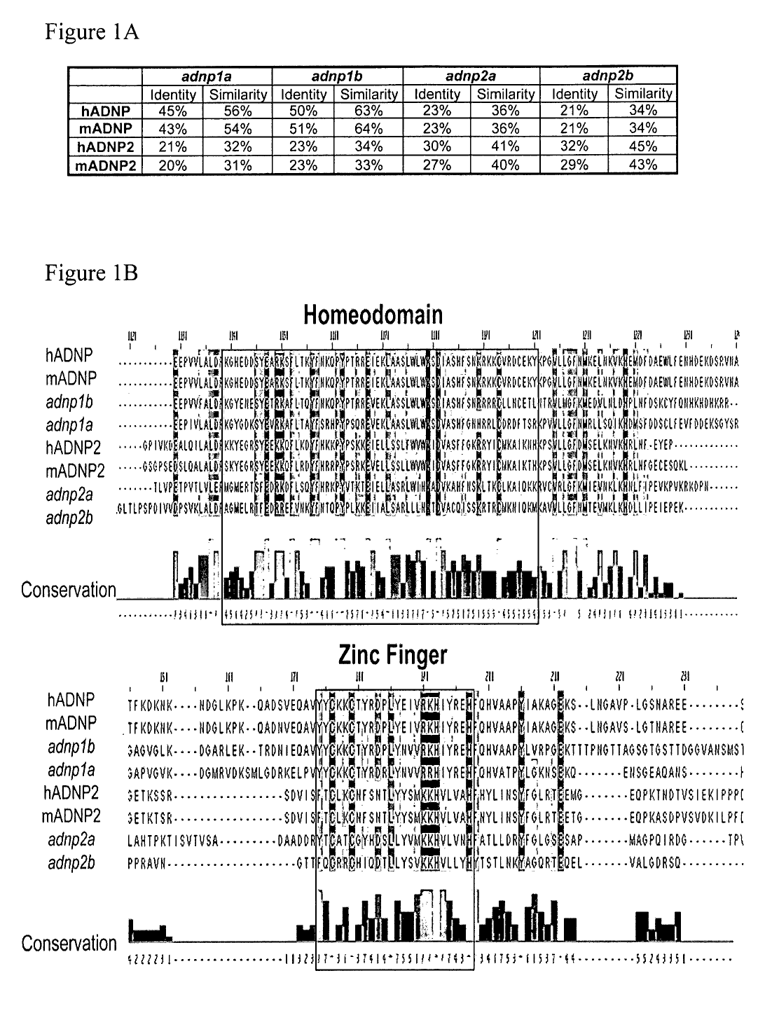

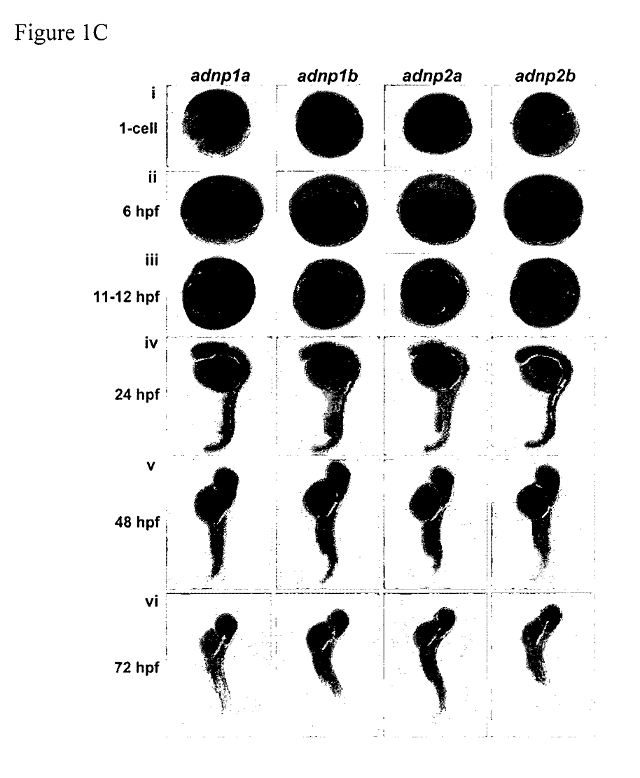

A BLAST search for human ADNP (NP_{—}056154) and ADNP2 (NP_{—}055728) orthologs in zebrafish revealed 2 zebrafish orthologs for each mammalian gene. adnp1a (NP_{—}001073484) and adnp1b (XP_{—}002666520), located on chromosome 11 and 23, respectively, are the 2 mammalian ADNP orthologs. adnp2a (NP_{—}001091735) and adnp2b (NP_{—}001073482), located on chromosomes 16 and 19, respectively, are the 2 mammalian ADNP2 orthologs. The existence of 2 zebrafish orthologs for each mammalian gene reflects partial genome duplication that occurred during early evolution of teleosts (Amores et al., 1998, Science 282, 1711-1714). Using ClustalW, the amino acid sequence of zebrafish adnp1 and adnp2 were aligned with the human and mouse orthologs. Relative identities and similarities (%) are summarized in FIG. 1A. High conservation levels were found, especially along the homeodomain and zinc fingers motifs which exist in all 4 zebrafish orthologs (FIG. 1B). Next, the expression profile of zebrafish adnp1 and adnp2 was examined by whole-mount in-situ hybridization (WMISH) on embryos from 1-cell to 72 hours post-fertilization (hpf). All zebrafish adnp1 and adnp2 transcripts were detected at the 1-cell stage, prior to the onset of zygotic expression which starts at ˜2.75 hpf (Kane and Kimmel, 1993, Development 119, 447-456), and are therefore maternally deposited. While expression of adnp1a and adnp2b was detected during all stages of embryonic development examined, the expression of adnp1b and adnp2a was detected at 1-cell stage, before the onset of zygotic expression, then, during gastrulation, at 6 hpf, no signal was detected, and at 11-12 hpf (segmentation) signal was detected again and was visible until 72 hpf (FIG. 1C). This pattern indicates that all 4 transcripts are maternally expressed, but later on, zygotic expression of adnp1a and adnp2b precedes that of adnp1b and adnp2a. In addition, at early developmental stages, adnp1 and adnp2 were ubiquitously expressed throughout the embryos, but as development progressed, expression was strongest at the anterior region of the embryos (FIG. 1C).

Silencing of Zebrafish adnp1 and adnp2 Results in Defective Hematopoiesis

The functional role of zebrafish adnp1 and adnp2 in embryonic development was assessed by knockdown (KD) experiments using specific morpholino (MO) modified antisense oligonucleotides. After injection, the phenotype of live embryos and larvae was monitored daily for 10 days. Standard control-MO injection did not cause any visible morphological abnormalities.

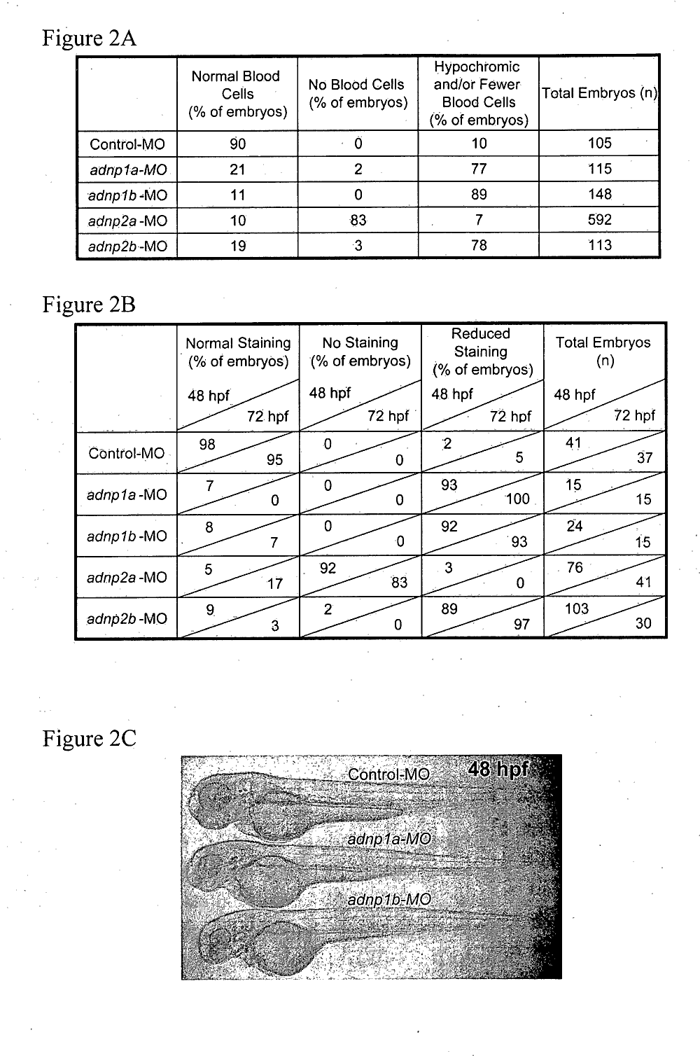

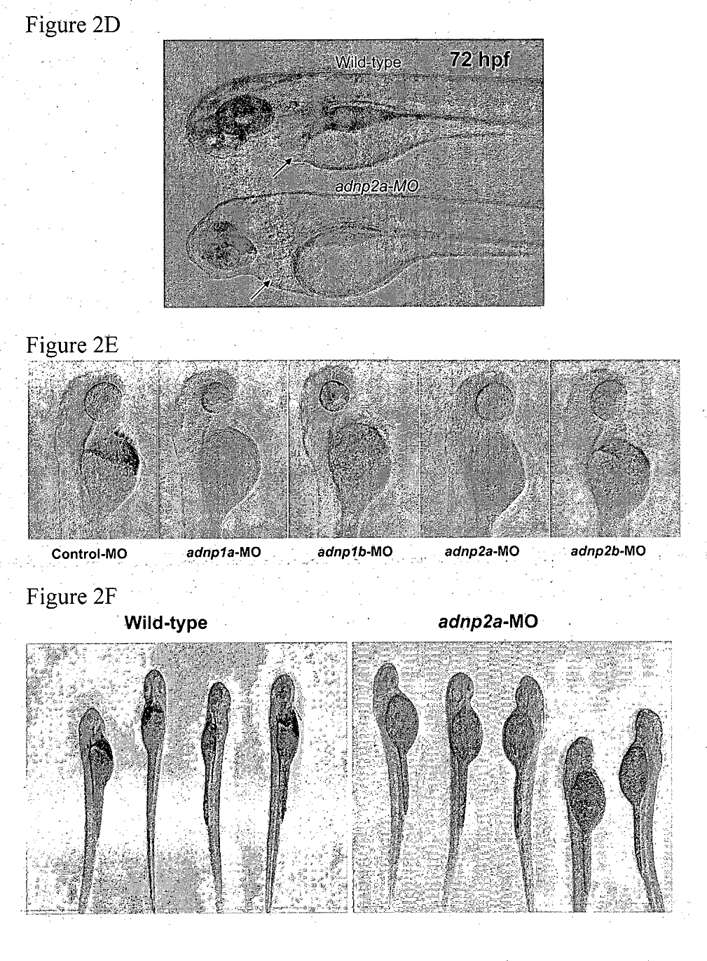

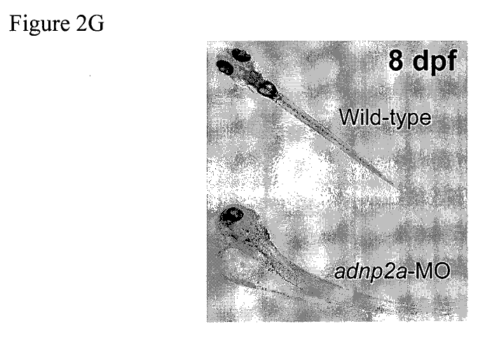

KD of adnp1a and adnp1b produced a similar phenotype: starting from 28 hpf, when blood circulation had already began, KD embryos had fewer blood cells in circulation than control-MO injected embryos. At 72 hpf, blood of adnp1a and adnp1b MO's-injected embryos was hypochromic compared to the red blood visible in control embryos (FIG. 2A). Hemoglobin (Hb) staining performed at 48 hpf and 72 hpf showed significant reduction in Hb content (FIGS. 2B and 2E). Cardiac edema was visible in some embryos. Regarding morphology, adnp1-KD embryos suffered from developmental delay, distorted tail morphology, and had much smaller head and eyes than control embryos (FIG. 2C, smaller head and eyes were seen in 73% of embryos, n=171 for adnp1a-MO and 92% of embryos, n=276 for adnp1b-MO compared with 0% of embryos, n=115 for control-MO). The efficiency of the adnp1a splicing MO was evaluated by RT-PCR. Silencing of adnp2b gave similar results regarding blood (FIGS. 2A, 2B, and 2E), although no developmental delay or major morphological changes were noted (however, injection of higher doses of adnp2b-MO did cause distorted morphology). While KD of adnp2a had no effect on the overall morphology, it exhibited the most dramatic effect on hematopoiesis. At the onset of blood circulation (˜24 hpf), blood cells were visible, however, between 33-48 hpf, no blood cells were evident and the embryos developed noticeable cardiac edema (FIGS. 2A and 2D). Staining for Hb was totally negative (FIGS. 2B, 2E and 2F). High magnification light microscopy revealed isolated blood cells occasionally moving across the vessels. It is worthwhile mentioning that some adnp2a-MO injected embryos recovered blood circulation at day 4 (ranging from 20% in some experiments to 70% in others). These results may indicate normal onset of the definitive wave of hematopoiesis (discussed below) or dilution and/or degradation of MO due to ongoing cell divisions. However, most of zebrafish adnp1 and adnp2 MO's-injected embryos did not resume blood circulation. Moreover, they became severely edematous at day 4-5 (FIG. 2G), no blood cells were visible in their vessels, and death occurred toward the end of the first week.

Maturation of the Erythroid Lineage During Zebrafish Primitive Hematopoiesis is Impaired in the Absence of adnp1 and adnp2

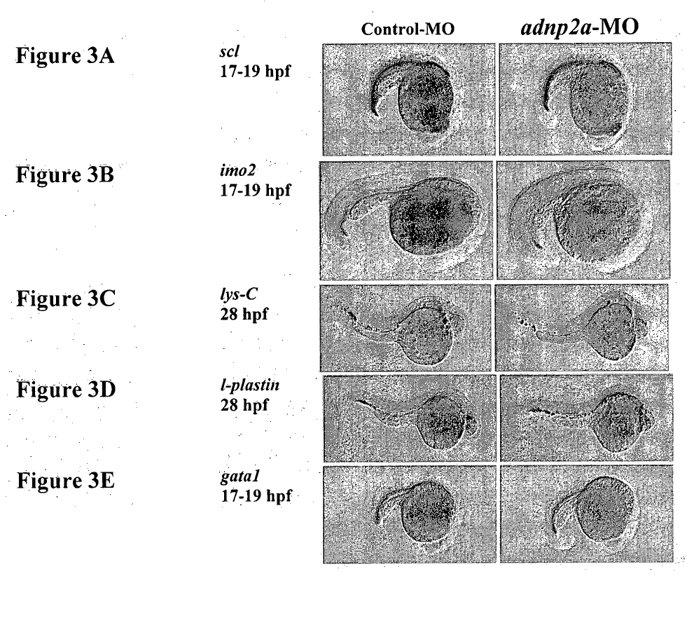

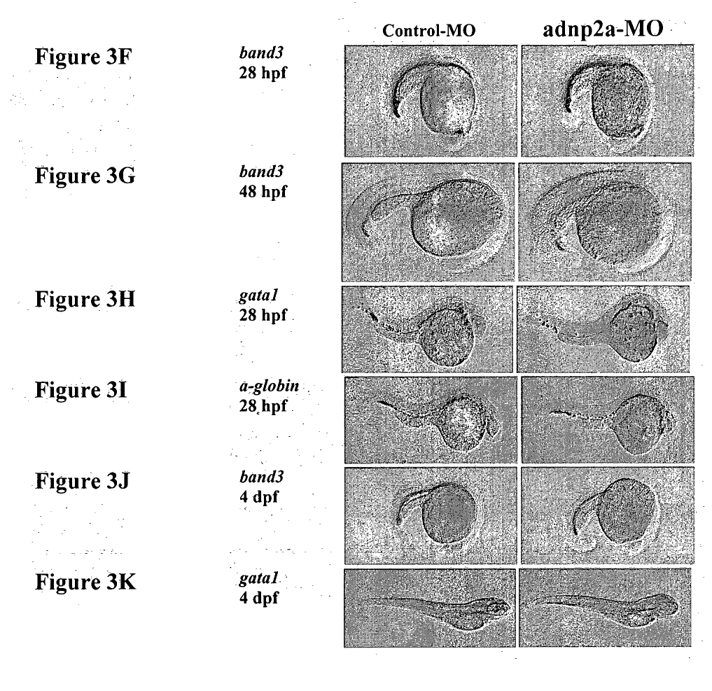

As the most dramatic effect on blood formation was achieved by knocking-down adnp2a, it was chosen for further phenotypic characterization. Zebrafish hematopoiesis occurs in 2 successive waves, ‘primitive’ and ‘definitive,’ as it does in other vertebrates (Galloway and Zon, 2003, Curr Top Dev Biol 53, 139-158). Stem cells of both waves are specified from the mesoderm layer. The primitive wave of hematopoiesis produces predominantly erythroid cells that begin to circulate at 24-26 hpf. The intermediate cell mass (ICM), located in the trunk, ventral to the notochord, is the site of primitive zebrafish erythropoiesis, and is analogous to the extraembryonic yolk sac blood islands of mammals. The second, definitive, wave of hematopoiesis is thought to initiate by ˜30-36 hpf in the ventral wall of the dorsal aorta, which is equivalent to the aorta-gonad-mesonephros in mammals. Definitive hematopoietic progenitors give rise to all hematopoietic cell lineages during the lifespan of the animal (Davidson and Zon, 2004, Oncogene 23, 7233-7246; McGrath and Palis, 2008, Curr Top Dev Biol 82, 1-22). The defects observed in hematopoiesis in this study occurred early during development in correspondence with the primitive wave (Liao et al., 2002, Development 129, 649-659). To identify the exact stage in which primitive hematopoiesis was blocked, the expression of several markers characteristic of specific stages during this process was examined (Juarez et al., 2005, Development 129, 649-659). lmo2 and scl are early hematopoietic stem cells markers, first expressed in zebrafish from the 1-3 somite stage (10-11 hpf) (Davidson and Zon, 2004, supra). At 17-19 hpf the expression of scl and lmo2 was intact in the ICM of adnp2a-MO injected embryos (100% of embryos, n=35 for scl and 100% of embryos, n=38 for lmo2), indicating that specification of the mesoderm layer to the hematopoietic lineage occurred normally (FIGS. 3A and 3B). Zebrafish, as opposed to mammals, undergo robust myelopoiesis during primitive hematopoiesis, with development of both granulocytes and macrophages. In order to evaluate whether the defect observed in hematopoiesis was specific to the erythroid lineage or appeared also in the myeloid lineage, the expression of 2 terminal myeloid markers was examined: lys-C which marks granulocytes (Su et al., 2007, Zebrafish 4, 187-199) and l-plastin which marks macrophages (Bennett et al., 2001, Blood 98, 643-651). At 28 hpf, expression of lys-C and l-plastin was intact in adnp2a KD embryos (100% of embryos, n=35 for lys-C and 100% of embryos, n=30 for l-plastin), meaning that myeloid differentiation was not affected (FIGS. 3C and 3D).

The development of the erythroid lineage was next examined. Surprisingly, expression of the erythroid marker gata1 (Davidson and Zon, 2004, supra) appeared normal in the ICM of adnp2a-MO injected embryos at 17-19 hpf (FIG. 3E, 100% of embryos, n=34) indicating that early commitment to the erythroid lineage was also normal. Although expression of gata1 demonstrated intact early erythroid development, the erythroid progenitors did not appear to develop into hemoglobinized erythrocytes, as shown by the lack of circulating blood cells and the lack of Hb staining in older embryos from the same experiment. At 28 hpf and 48 hpf, expression of band3, an erythrocyte membrane protein crucial for erythrocytes maturation (Paw et al., 2003, Nat Genet. 34, 59-64), was almost absent in adnp2a-MO injected embryos compared to control embryos (FIGS. 3F and 3G, 83% of embryos, n=23 for 28 hpf versus 0% of embryos, n=18 for control-MO and 97% of embryos, n=30 for 48 hpf versus 7% of embryos, n=27 for control-MO). In addition, gata1 expression which was intact at 17-19 hpf was dramatically reduced in KD embryos at 28 hpf (FIG. 3H, 90% of embryos, n=21 versus 0% of embryos, n=26 for control-MO). Expression of embryonic α-globin 1, additional erythrocyte marker (Brownlie et al., 2003, Dev Biol 255, 48-61), was also significantly reduced at 28 hpf in adnp2a KD embryos (FIG. 3I, 96% of embryos, n=24 versus 0% of embryos, n=23 for control-MO). At 4 days post-fertilization (dpf), gata1 and band3 expression were almost absent in control embryos, representing erythroid cell maturation (Brownlie et al., 1998, Nat Genet. 20, 244-250), while in adnp2a-MO injected embryos ectopic expression of gata1 and band3 was visible along the tail and yolk sinus (FIGS. 3J and 3K, 86% of embryos, n=21 versus 0% of control-MO, n=17 for gata1 and 100% of embryos, n=15 versus 0% of control-MO, n=15 for band3), suggesting blocked erythroid maturation as a result of adnp2a suppression.

Regarding the definitive wave of hematopoiesis and vasculogenesis, preliminary WMISH data indicated no involvement of adnp2a in the definitive wave and a possible effect on vasculogenesis.

The Expression of scl at 17-19 hpf and of lys-C, gata1, band3 and α-globin at 28 hpf was also evaluated for the 3 other MO's (adnp1a-MO, adnp1b-MO, adnp2b-MO). Results were similar to those obtained with adnp2a-MO, although less robust.

To summarize, silencing of zebrafish adnp1 and adnp2 resulted in defective primitive erythropoiesis. While hematopoietic specification from the mesoderm and myeloid differentiation was not affected, there was a complete block in erythroid maturation.

### Expression of ADNP and ADNP2 is Required for Proper Erythroid Differentiation in Mouse Erythroleukemia Cells

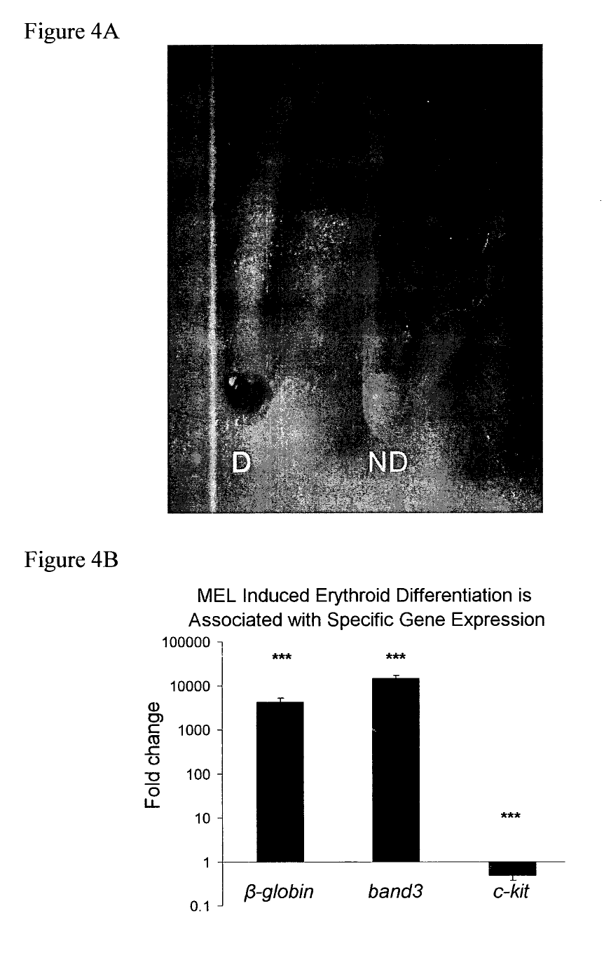

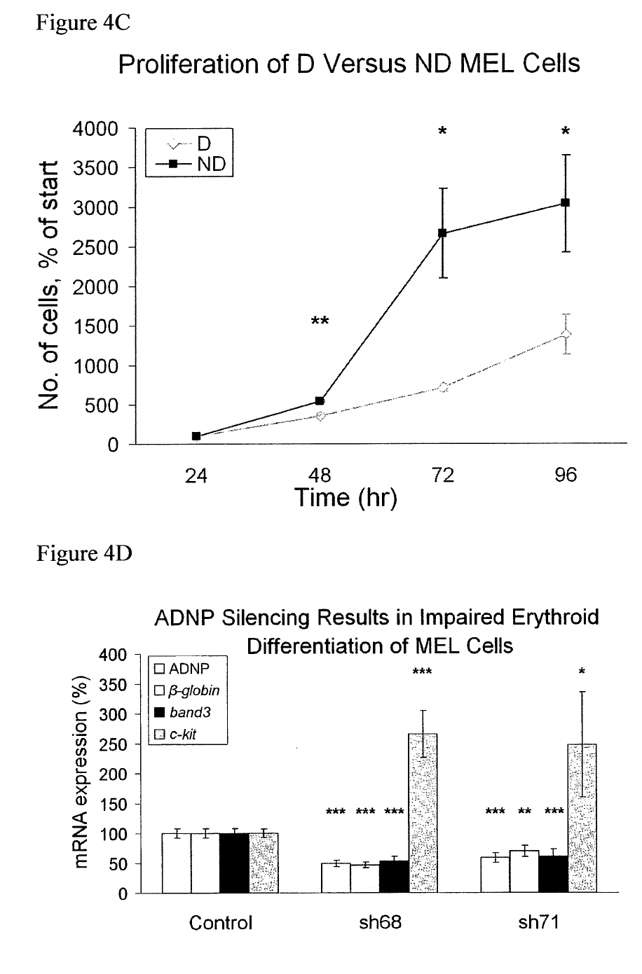

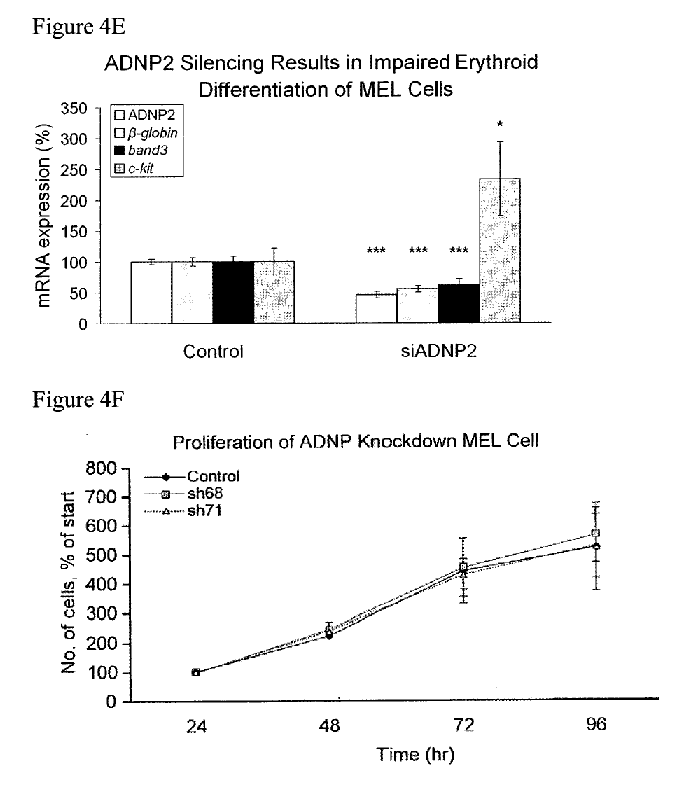

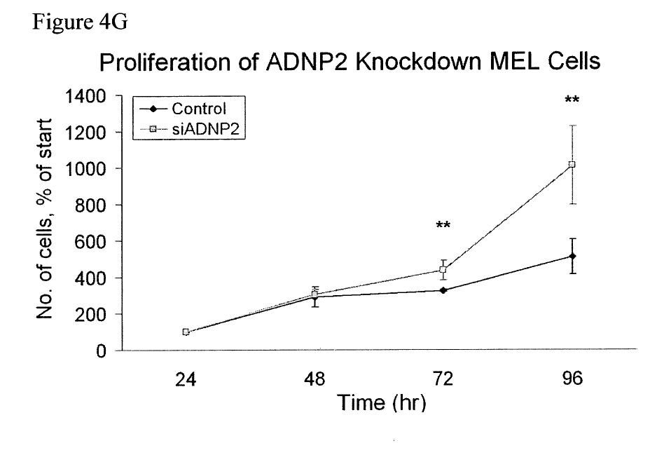

To test whether the role of ADNP and ADNP2 in zebrafish erythroid maturation is conserved in mammals, the inventors used the MEL model, one of the best in vitro models for erythroid differentiation. MEL cells are Friend-virus transformed erythroid precursors blocked at a relatively early stage in the pathway of differentiation. To induce differentiation, cells were treated with 2% DMSO for 4 days (Friend et al., 1971, Proc Natl Acad Sci USA 68, 378-382; Orkin, 1978, In Vitro 14, 146-154) resulting in red cell pellets due to Hb production, compared with the white pellet of control non-differentiated cells (ND, FIG. 4A). To ascertain effective erythroid differentiation followed by DMSO treatment, 2 parameters were evaluated: mRNA expression of specific erythroid markers and growth rate. As expected, the expression level of β-globin and band3 (Sabban et al., 1980, J Cell Physiol 104, 261-268), specific erythroid markers, was dramatically increased by 1000 or 10000 fold respectively in the DMSO-treated cells. The expression of c-kit, marker of hematopoietic stem cells representing undifferentiated cells (Uoshima et al., 1995, Br J Haematol 91, 30-36), was 200-times lower in the DMSO-treated cells compared with ND cells (FIG. 4B). Growth rate of differentiating (D) cells showed a significant reduction compared with ND cells (FIG. 4C), corresponding to the growth arrest accompanying cell differentiation processes.

To test the effect of ADNP and ADNP2 silencing on differentiation, cells were transfected with 2 different ADNP shRNA plasmids (sh68 and sh71) or with ADNP2 siRNA prior to addition of 2% DMSO. The efficiency of silencing after 4-days DMSO treatment was evaluated at the RNA level (40-50% silencing for ADNP and 55% for ADNP2) and protein level. Analysis of specific erythroid markers by Real-Time PCR indicated that ADNP silencing resulted in 30-55% reduction in β-globin mRNA expression, 40-50% reduction in band3 and ˜250% increase in c-kit expression compared with control cells treated with non-specific shRNA plasmid (FIG. 4D). Similarly, ADNP2 KD reduced β-globin expression by 45%, band3 by 40% and increased c-kit expression by ˜250%, compared with control cells treated with scrambled ADNP2 siRNA sequences (FIG. 4E). Cells treated with ADNP2 siRNA were 2-fold more proliferative than control cells at the 4th experimental day (FIG. 4G), indicating that the balance between differentiation and proliferation was in favor of proliferation in the ADNP2 siRNA treated cells. In contrast, ADNP silencing did not affect cell proliferation (FIG. 4F), although it down-regulated β-globin and band3 expression.

### Exogenous ADNP/ADNP2 RNA Rescues the Undifferentiated Phenotype Caused by ADNP/ADNP2 Silencing

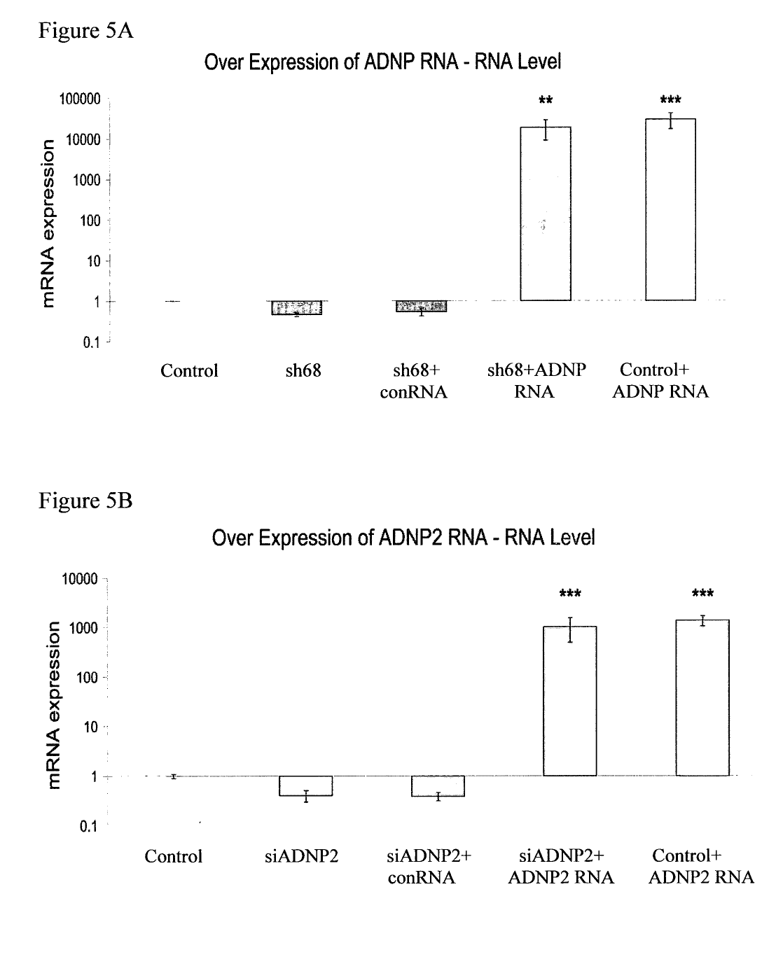

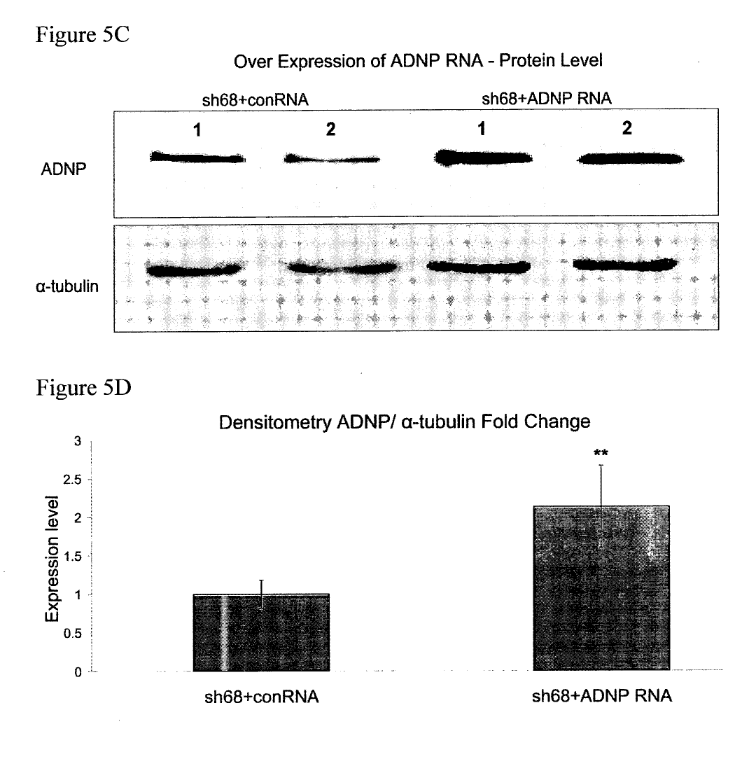

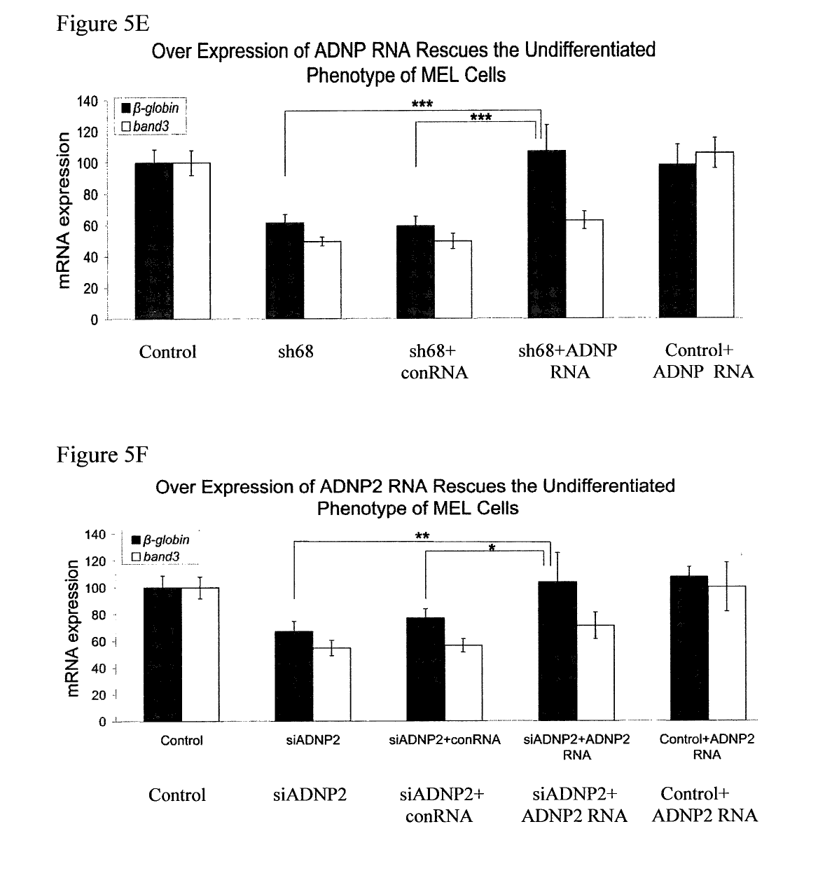

In order to prove that the failure of MEL cells to differentiate was specific to ADNP and ADNP2 silencing, rescue experiments were performed with RNA encoding mouse ADNP/ADNP2. While addition of control Xenopus RNA did not affect ADNP or ADNP2 expression levels 4 days after transfection, addition of ADNP/ADNP2 RNA to ADNP/ADNP2 KD samples or to control samples (treated with non-specific siRNA/shRNA sequences) resulted in 10000 or 1000 times increase in ADNP and ADNP2 expression level, respectively, (FIGS. 5A and 5B). Thus, exogenous RNA was efficiently transfected and was not destroyed by the RNAi machinery, probably due to its high concentration. Next, it was confirmed that the exogenous RNA was efficiently translated. 4 days after transfection, total protein was extracted from cells treated with ADNP shRNA (sh68)+control RNA and from cells treated with ADNP shRNA (sh68)+ADNP RNA, and evaluated by western analysis. FIGS. 5C and D show that the ADNP signal was significantly higher (2-fold) in cells transfected with ADNP RNA compared with cells transfected with control RNA.

To test whether ADNP/ADNP2 exogenous RNA would rescue the undifferentiated phenotype, ADNP or ADNP2 KD experiments were performed as above with the addition of exogenous ADNP/ADNP2 RNA respectively. While addition of control Xenopus RNA had no significant effect on β-globin or band3 levels, addition of RNA encoding ADNP or ADNP2 completely rescued β-globin levels to control levels (FIGS. 5E and 5F). band3 levels were slightly increased as a result of the expression of ADNP and ADNP2 RNA, but this trend was not significant. Addition of ADNP or ADNP2 RNA to controls cells, treated with non-specific siRNA/shRNA, had no effect on β-globin or band3 expression level, indicating that ADNP/ADNP2 RNA affected only KD cells in which the differentiation process was damaged, but not control cells in which differentiation was intact (FIGS. 5E and 5F).

### Brg1, a Component of the SWI-SNF Chromatin Remodeling Complex, is Possibly Involved in Mediating the Effect of ADNP and ADNP2 on MEL Cells Differentiation

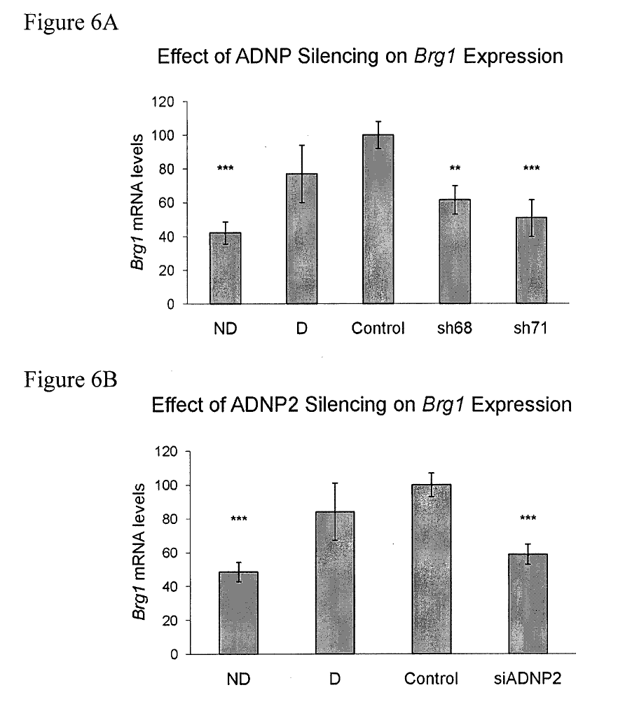

Brg1 is involved in the hematopoietic process through regulation of the β-globin locus (Bultman et al., 2005, Genes Dev 19, 2849-2861; Griffin et al., 2008, Development 135, 493-500). In addition, one of the inventors has previously shown that ADNP interacts with Brg1 (Mandel and Gozes, 2007, supra). Thus, it was assumed that Brg1 expression would be influenced by ADNP and ADNP2 silencing. As demonstrated in FIG. 6, Brg1 levels in ND cells were ˜2 times lower than the levels in differentiated (D) cells, implying that Brg1 was associated with erythroid differentiation in MEL cells. In ADNP and ADNP2 KD cells, Brg1 levels were 40-50% lower than those in control cells treated with non-specific siRNA/shRNA sequences. Interestingly, while Brg1 levels in control cells were quite similar to the levels in D cells, Brg1 levels in ADNP and ADNP2 KD cells were similar to the levels in the ND cells. Thus, Brg1 may mediate in part the effect of ADNP and ADNP2 on MEL cells differentiation.

### Discussion

This study reveals for the first time a crucial role for ADNP and ADNP2, each separately, in globin synthesis and erythropoiesis in two model systems; the zebrafish and the murine erythroleukemia (MEL) cells.

The present inventors found that zebrafish adnp1 and adnp2 are ubiquitously expressed during the first 3 days of development, a time period that is associated with rapid embryogenesis and organogenesis (Kimmel et al., 1995, Dev Dyn 203, 253-310). Using MO knockdown experiments, the inventors have demonstrated that all 4 zebrafish adnp1 and adnp2 genes are required for primitive erythropoiesis. These data indicate that maturation and differentiation of the erythroid lineage was the main defect caused by adnp1 and adnp2 silencing and was probably the reason for the reduced number of circulating cells and to the block in Hb synthesis. Mesodermal specification to the hematopoietic lineage, myeloid differentiation and early erythroid development were not affected by ADNP/ADNP2 silencing. Definitive hematopoietic markers appeared normal in adnp2a KD embryos and part of the embryos resumed blood circulation at 4 dpf. As the “bloodless” mutant (that has restricted defect in the primitive wave but not in the definitive wave of hematopoiesis), resumed its blood circulation at around 5 dpf (Liao et al., 2002, supra), these results may indicate an undamaged definitive wave. However, since not all adnp2a KD embryos resumed their blood circulation and almost none of the adnp1a, adnp1b and adnp2b KD embryos resumed blood circulation, further experiments are required in order to draw firm conclusions regarding the definitive wave. In this context, in MEL cells, terminal differentiation closely resembles definitive erythropoiesis, since it results in the expression of adult type globin genes, and in these cells ADNP and ADNP2 expression was required for globin expression. It is possible that there is a compensatory effect in the KD zebrafish by other members of the ADNP family in the case of the adnp2a. It is also possible that a complete knockout is required for a non-reversible phenotype.

In MEL cells, silencing of ADNP and ADNP2 resulted in defective erythroid differentiation, characterized by down-regulation of the erythroid markers β-globin and band3 and up-regulation of the hematopoietic stem cells marker, c-kit. These results corroborated the inventors' observation in the zebrafish model. Expression of exogenous RNA encoding ADNP and ADNP2 rescued the undifferentiated phenotype of MEL cells by up-regulating β-globin levels back to control levels, strongly supporting the specificity of ADNP and ADNP2 knockdowns.

Primitive erythropoiesis in the mouse is first observed on E7.5 in blood islands of the yolk sac. These primitive cells are the exclusive red cells in the embryo until the newly formed fetal liver releases the first definitive red cells into the circulation at E12. Therefore, anemia observed in the fetus before E13 is due to loss or decreased synthesis of primitive erythroid cells (McGrath and Palis, 2008, supra). ADNP knockout (KO) mouse embryos die between E8.5-E9.5, a time period corresponding to primitive hematopoiesis, which was also impaired in the zebrafish embryos. Interestingly, fgf1, lhx2, hipk2, flt1 and klf2 that were down-regulated in the ADNP-KO mice (Mandel et al., 2007, supra) were shown to be required for proper hematopoiesis (Basu et al., 2005, Blood 106, 2566-2571; Fong et al., 1995, Nature 376, 66-70; Hattangadi et al., 2010, Blood 115, 4853-4861; Porter et al., 1997, Development 124, 2935-2944; Songhet et al., 2007, Dev Dyn 236, 633-643). The inventors suggest that hematopoietic abnormalities accompanied the neural tube closure defect in the ADNP-KO mice and presented the underlining cause for their early death. This is in-line with Copp's findings (Copp, 1995, Trends Genet. 11, 87-93) that many embryonic organs and body systems have little or no survival value in uteri, while hematopoiesis defects are the leading cause for in-uteri death in mouse embryos between E2-E15. Failure to maintain a functioning yolk sac circulation was observed in many embryos that died during early organogenesis, the time-period in which ADNP-KO mice have died (Copp, 1995, supra).

While ADNP-KO mice died at early developmental stages, the KD zebrafish embryos survived their first week of life. It should be taken into consideration that there is a higher redundancy in the zebrafish (4 ADNP gene family in the zebrafish compared to 2 in the mouse), and that in the mouse, ADNP knockout was complete, while in zebrafish ADNP was knocked down. Furthermore, previous studies demonstrated that completely anemic zebrafish mutants are viable for at least 2 weeks post-fertilization, suggesting that blood cell mediated oxygen transport is not critical until relatively late stages of larval development (Pelster and Burggren, 1996, Circ Res 79, 358-362).

Previously, the present inventors have demonstrated that ADNP interacts with Brg1, member of the SWI/SNF chromatin remodeling complex (Mandel and Gozes, 2007, supra), that coordinates the disruption of nucleosomes to permit the binding of various transcription factors, an activity crucial for achieving the correct spatiotemporal gene expression during embryonic development (de la Serna et al., 2006, Nat Rev Genet. 7, 461-473). In this study the inventors showed that MEL cells failure to differentiate is associated with reduced Brg1 mRNA expression. Moreover, Brg1 expression level in ADNP/ADNP2 KD cells resembled that of ND cells. Previous studies have demonstrated that Brg1 is involved in hematopoietic regulation during both primitive and definitive erythropoesis. It was found that Brg1 mediates chromatin remodeling of the β-globin locus and is required for transcription of both embryonic and adult globins (Bultman et al., 2005, supra; Griffin et al., 2008, supra). It was further shown that Brg1 is recruited to the β-globin locus by selective association with zinc finger transcription factors such as EKLF and gata1, but not with other transcription factors that do not contain zinc finger domains (Kadam and Emerson, 2003, Mol Cell 11, 377-389). ADNP and ADNP2 also belong to the zinc-finger protein family. It is thus postulated that their involvement in erythropoiesis is mediated through recruitment of Brg1 to the β-globin locus. Interestingly, silencing of ADNP and ADNP2 in MEL cells resulted in down-regulation of β-globin and band3, paralleling the reduced band3 levels in erythroblasts of conditional Brg1 knockouts mice (Griffin et al., 2008, supra).

ADNP and ADNP2 are homologous proteins, but their exact interrelationship remains to be further elucidated. In zebrafish hematopoiesis, there was high resemblance between the phenotype of adnp1 and adnp2 KD embryos, yet it was not exactly the same. In this manner, KD of adnp2a caused complete lack of blood and Hb synthesis, while KD of the 3 other genes resulted in less severe phenotypes. More efficient gene KD achieved by adnp2a-MO might account for the increased phenotype severity. Alternately, redundancy between the 4 zebrafish adnp1 and adnp2 genes may exist. In MEL cells, either ADNP or ADNP2, each separately, were required for proper differentiation and for up-regulation of specific erythrocytes markers, but only ADNP2 affected cell growth during differentiation. Thus, ADNP and ADNP2 may have similar roles in some aspects but may have different regulatory effect on target genes, resulting in different functional outcomes.

In this work the present inventors focused on the hematopoietic defect, since it constituted the most prominent phenotype in the zebrafish embryos, and reflected an unexpected and exciting novel role for the ADNP protein family. However, additional changes were observed, especially in the adnp1 KD embryos. It was previously shown that ADNP is necessary for brain development (Pinhasov et al., 2003, Brain Res Dev Brain Res 144, 83-90) and regulates genes associated with neurogenesis (Mandel et al., 2007, supra). The present inventors observed smaller head and eyes in adnp1 MO's-injected embryos, and the preliminary data suggest a developmental delay in these embryos, showing delayed expression pattern of the CNS marker irx7.

To summarize, the present inventors have shown that the ADNP protein family is required for proper erythropoiesis. In the zebrafish model, adnp1 and adnp2 were not required for hematopoietic stem cells specification, myeloid differentiation or early erythroid development. Instead, they were required for progression through the later stages of erythroid differentiation. Similar results were seen in a mammalian model system, suggesting that this role is probably an ancestral function common to all vertebrate ADNP and ADNP2 genes. MEL cells failure to differentiate in the absence of ADNP or ADNP2 was associated with reduced Brg1 expression. It is suggested that the effect of ADNP and ADNP2 on erythroid development is achieved in part through Brg1 recruitment to the promoter of genes essential for hematopoiesis, such as β-globin. Finally, the present inventors were the first to examine the developmental function of ADNP2 in vivo, and to reveal a novel, striking role for ADNP and ADNP2 in erythropoiesis regulation. The findings of this study add another dimension to the understanding of the complexity in regulating embryogenesis in general and erythropoiesis in particular, by the ADNP protein family.

TABLE 1
Primer Pairs in Reverse Transcription and
Quantitative Real Time PCR

| Primer | Sequence |
|---|---|
| Mouse ADNP | 5′-acgaaaaatcaggactatcgg-3′ |
| (NM_009628) | 5′-ggacattccggaaatgacttt-3′ |
| Mouse ADNP2 | 5′-ggaaagaaagcgagataccg-3′ |
| (NM_175028) | 5′-tcctggtcagcctcatcttc-3′ |
| Mouse β-globin (Hbb-b1) | 5′-gcaggctgctggttgtct-3′ |
| (NM_008220) | 5′-catgggccttcactttgg-3′ |
| Mouse band3 (Slc4a1) | 5′-tgcttgtgggactgtccat-3′ |
| (NM_011403) | 5′-aagagctggatgccactgag-3′ |
| Mouse c-kit | 5′-caaaggaaatgcacgactgc-3′ |
| (NM_001122733) | 5′-tcccataggaccagacatca-3′ |
| Mouse Brg1 (Smarca4) | 5′-gcaggatgaggaggaagatga-3′ |
| (NM_011417) | 5′-gcgcatgaagaggtcaaact-3′ |
| Mouse 18S ribosomal | 5′-cctgcggcttaatttgactc-3′ |
| RNA (NR_003278) | 5′-aactaagaacggccatgcac-3′ |

### Example 2

### Detection of ADNP-Like Immunoreactivity in Milk Samples

### Methods

Powdered milk (DIFCO™, skim milk, Becton, Dickenson and Company, Sparks Md., USA) was dissolved in water and evaluated by Bradford-Bio-Rad protein assay (Munchen, Germany) and increasing amounts were separated on polyacrylamide gels followed by western blot analysis using ADNP antibody (Zamostiano et al., 2001, supra) as follows.

Proteins were separated by electrophoresis on a 10% (w/v) polyacrylamide gel containing 0.1% SDS. Molecular weights were determined using Precision Plus Protein Standards (Dual Color, BioRad, Hercules, Calif., USA). Following electrophoresis, proteins were transferred to nitrocellulose membranes (Whatman Plc., Kent, UK) and nonspecific antigen sites were blocked using a solution containing 5% bovine serum albumin (w/v) in TBST (10 mM Tris pH 8, 150 mM NaCl and 0.05% Tween 20). Antigen detection was performed over-night at 4° C. using the ADNP antibody cited below (diluted 1:200). Antibody-antigen complexes were detected using horseradish peroxidase-conjugated goat anti-rabbit IgG secondary antibody (Jackson ImmunoResearch, West Grove, Pa., USA) (1 hours incubation at room temperature) and visualized by chemiluminescent kit (Pierce).

The specifications of the primary antibody were as follows:

ADNP Antibody Catalog No. A300-104A (Bethyl Laboratories, Montgomery, Tex., USA) Produced in Rabbit
Amount: 0.1 ml at 1 mg/ml
Buffer: Tris-citrate/phosphate buffer, pH 7 to 8 containing 0.1% Sodium Azide
Shelf Life: 1 year from date of receipt
Production Procedures Antibody was affinity purified using an epitope specific to ADNP immobilized on solid support. The epitope recognized by A300-104A maps to a region between residues 1050 and the C-terminus (residue 1102) of human Activity-Dependent Neuroprotective Protein using the numbering given in entry NP_{—}056154.1 (GeneID 23394). Immunoglobulin concentration was determined by extinction coefficient: absorbance at 280 nm of 1.4 equals 1.0 mg of IgG.

### Results

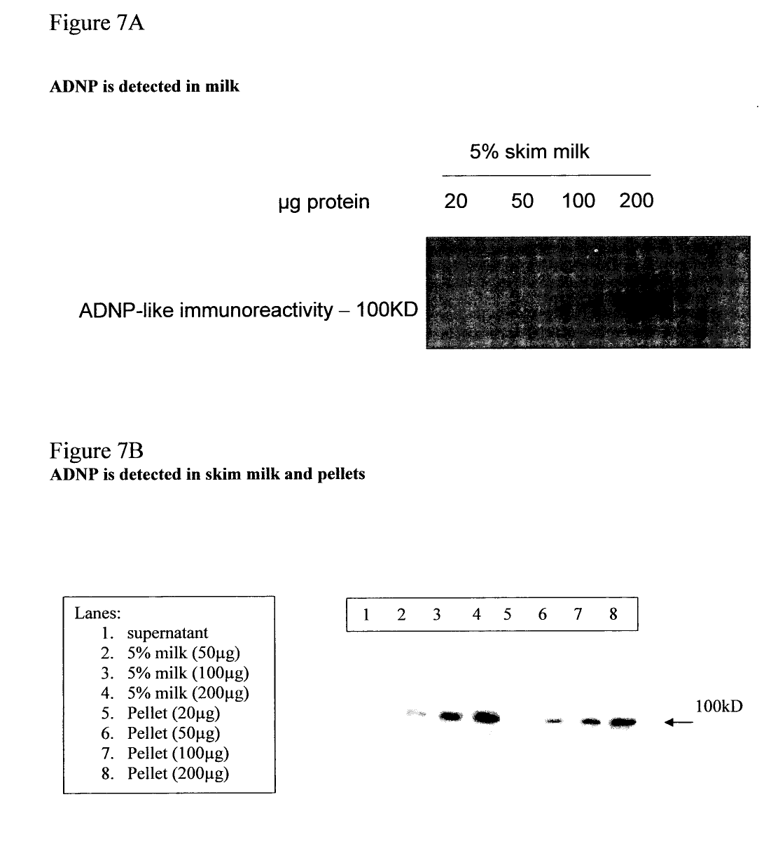

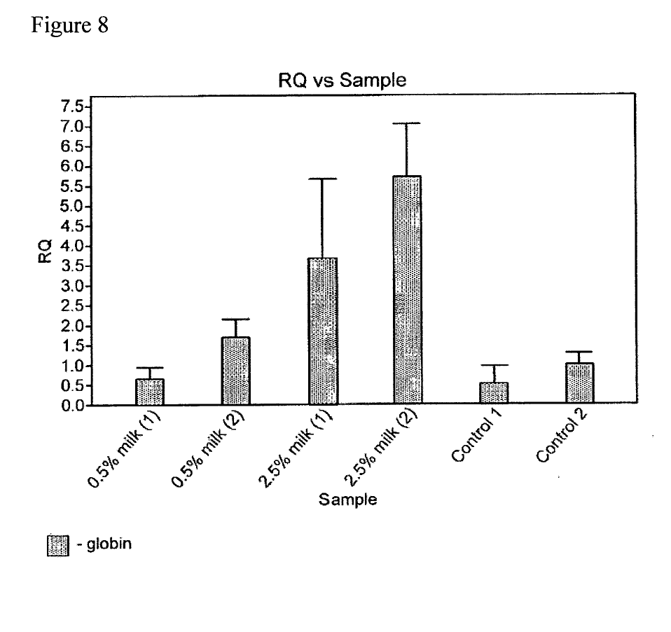

ADNP was detected in milk. As shown in FIG. 7A, milk protein (20-200 μg/lane) were loaded on the gel and ADNP-like immunoreactivity (−100 KD) was detected in the milk samples, the intensity of immunoreactivity correlated with the amount of the milk protein. Further studies used 8000×g centrifugation for 1 hr of 5% skim milk (1 ml fractions). Supernatants (1 ml) were collected and pellets dissolved in 1 ml water before electrophoresis. ADNP quantity was shown in FIG. 7B to be present in milk pellets and to correlate with the amount of milk protein.

### Discussion

ADNP has cellular import and export sequences (Gozes et al., 2000, Ann N Y Acad Sci, 921, 115-118; Zamostiano et al., 2001, supra; Furman et al., 2004, Neuron Glia Biology, 1, 193-199) and as ADNP immunoreactivity is found in milk it can provide regulation of globin synthesis. ADNP-like immunoreactivity seems to segregate into the milk pellets, casein micelles that may be considered as potential carriers (Al-Ghobashy et al., 2009, J Chromatogr B Analyt Technol Biomed Life Sci, 877, 1667-1677). ADNP-like activity in milk products makes diary products useful in an anti-anemia therapy.

Furthermore, several approaches employing casein micelle disruption have been tried: (i) isoelectric point precipitation of caseins using 1.0M acetic acid to pH 4.6 and solubilization of the casein pellet using 8.0 Murea, (ii) disruption of the casein micelles by addition of either urea or arginine to the milk samples to a final concentration of 8.0 and 2.0M respectively, and (iii) selective precipitation of caseins using calcium phosphate nanoparticles (CAP) as previously described [Ibid and Morcol, T., He, Q. & Bell, S. J. Model process for removal of caseins from milk of transgenic animals. Biotechnol Prog 17, 577-582 (2001)]. Association of ADNP with the casein micelle (FIG. 2B), may increase its bioavailability, and may allow oral application and also increase brain bioavailability.

### Example 3

### Milk Enhances Globin Formation

### Methods

### Murine Erythroleukemia Cell Line

The murine erythroleukemia cell line (MEL) was cultured at 37° C. in 5% CO₂ incubator in RPMI-1640 medium supplemented with 10% fetal calf serum, 2 mM L-glutamine, 100 U/ml penicillin, 0.1 mg/ml streptomycin and 12.5 U/ml nystatin (Biological Industries, Beit Haemek, Israel). Due to their ability to differentiate into hemoglobin producing cells, these cells were chosen as the model system in this study.

### Milk Addition

To test the ability of milk to enhance globin formation, incubation with increasing milk concentrations was carried out. 5×10⁴ cells were plated in 500 μl growth medium on a 24 well plate, and treated with one of the four following treatments for 4 days: 1) 2.5% milk; 2) 0.5% milk; 3) control (non-treated cells). Each sample was repeated twice and measured thrice by Real-Time PCR. On the fourth day of incubation, the cells were harvested and RNA was extracted. Real-Time PCR analysis was then carried out in order to evaluate β globin mRNA expression.

### RNA Extraction

Total RNA from MEL cells was extracted using the RNeasy Plus Mini Kit (Qiagen, Hilden, Germany) according to the manufacture protocol. The quantity of RNA was determined by measuring OD260 with a NanoDrop spectrophotometer (NanoDrop Technologies, Wilmington, Del., USA).

### Reverse Transcription and Quantitative Real Time PCR

Samples with same amount of total RNA were used to synthesize single-strand cDNA using the Applied Biosystems High Capacity cDNA Reverse Transcription Kit (Applied Biosystems, Foster City, Calif., USA) and random hexamers primers according to the manufacturer's instructions. In each RT-PCR run, two negative controls were included: sterile water to control for the purity of the reagents and total RNA without the RT enzyme to test for genomic contamination. Primer pairs (provided in Table 2) were designed using the primer 3 web interface (website: frodo.wi.mit.edu/primer3/) and synthesized by Sigma-Genosys (The Woodlands, Tex., USA). To avoid amplification of contaminating genomic DNA, primers were designed to be located at the exon-exon junction.

Real Time PCR was performed using the SYBR® Green PCR Master Mix and StepOnePlus instrument and software (Applied Biosystems) using the default thermocycler program: 10 minutes of pre-incubation at 95° C. followed by 40 cycles for 15 seconds at 95° C. and one minute at 60° C. Real-time PCR reactions were carried out in a total volume of 10 μl in a 96-well plate (Applied Biosystems) containing 5 μl of X2 SYBR® Green PCR Master Mix and 250 nM of each sense and antisense primers. The comparative Ct method was used for quantification of transcripts. 18S ribosomal RNA was used as the normalization control gene.

TABLE 2

| | |
|---|---|
| Mouse β-globin (Hbb-b1) | 5′-gcaggctgctggttgtct-3′ |
| (NM_008220) | 5′-catgggccttcactttgg-3′ |
| Mouse 18S ribosomal RNA | 5′-cctgcggcttaatttgactc-3′ |
| (NR_003278) | 5′-aactaagaacggccatgcac-3′ |

### Results

Results showed that incubation with increasing milk concentrations led to increased globin synthesis. This indicates the usefulness of dairy products in treating anemia.

All patents, patent applications, and other publications, including GenBank Accession Numbers, cited in this application are incorporated by reference in the entirety for all purposes.

1. A method for treating anemia, comprising administering a therapeutically effective amount of an ADNP polypeptide to a subject in need thereof, wherein the ADNP polypeptide is:
(a) a full length ADNP polypeptide (SEQ ID NO:1);
(b) a fragment of the full length ADNP polypeptide comprising an active core site of PGVLL (SEQ ID NO:6), ARKS (SEQ ID NO:7), SDIASHFSNKRKKCVR (SEQ ID NO:8), or KRTYEQMEFPLLKKRKLD (SEQ ID NO:9); or
(c) a polypeptide comprising the full length ADNP polypeptide of (a) or the fragment of (b) and having up to about 40 amino acids at either or both of the N-terminus and the C-terminus.

2. The method of claim 1, wherein the fragment comprises an active core site of EARKSFLTKYFNKQPYPTRREIEKLAASLWLWKSDIASHFSN-KRKKCVRDCEKYKPGVLLGFN (SEQ ID NO:2).

3. The method of claim 1, wherein the ADNP polypeptide is a full length ADNP polypeptide (SEQ ID NO:1).

4. (canceled)

5. The method of claim 1, wherein the ADNP polypeptide consists of SEQ ID NO:2.

6. The method of claim 1, wherein the active core site of the ADNP polypeptide comprises at least one D-amino acid.

7. A method for treating anemia, comprising administering a therapeutically effective amount of an ADNP2 polypeptide to a subject in need thereof, wherein the ADNP2 polypeptide is:
(a) a full length ADNP2 polypeptide (SEQ ID NO:3);
(b) a fragment of the full length ADNP2 polypeptide comprising an active core site of PSVLL (SEQ ID NO:10), EKKQ (SEQ ID NO:11), or IDVASFFGKRRYICMK (SEQ ID NO:12); or
(c) a polypeptide comprising the full length ADNP2 polypeptide of (a) or the fragment of (b) and having up to about 40 amino acids at either or both of the N-terminus and the C-terminus.

8. The method of claim 7, wherein the fragment comprises an active core site of EEKKQFLKDYFHKKPYPSKKEIELLSSLFWVWKIDVASFFGKRRYIC-MKAIKNHKPSVLLGFD (SEQ ID NO:4).

9. The method of claim 7, wherein the ADNP2 polypeptide is the full length ADNP2 polypeptide (SEQ ID NO:3).

10.-11. (canceled)

12. The method of claim 7, wherein the active core site of the ADNP2 polypeptide comprises at least one D-amino acid.

13.-23. (canceled)

24. A method for treating anemia, comprising administering a therapeutically effective amount of an ADNF polypeptide to a subject in need thereof, wherein the ADNF polypeptide is a member selected from the group consisting of:
(a) an ADNF I polypeptide comprising an active core site having the amino acid sequence of Ser-Ala-Leu-Leu-Arg-Ser-Ile-Pro-Ala (SEQ ID NO:13);
(b) an ADNF III polypeptide comprising an active core site having the amino acid sequence of Asn-Ala-Pro-Val-Ser-Ile-Pro-Gln (SEQ ID NO:14); and
(c) a mixture of the ADNF I polypeptide of part (a) and the ADNF III polypeptide of part (b).

25. The method of claim 24, wherein the ADNF polypeptide is a member selected from the group consisting of a full length ADNF I polypeptide, a full length ADNF III polypeptide, and a mixture of a full length ADNF I polypeptide and a full length ADNF III polypeptide.

26. The method of claim 24, wherein the ADNF polypeptide is an ADNF I polypeptide.

27. The method of claim 26, wherein the active core site of the ADNF I polypeptide comprises at least one D-amino acid.

28. The method of claim 26, wherein the ADNF I polypeptide has the formula (R¹)ₓ-Ser-Ala-Leu-Leu-Arg-Ser-Ile-Pro-Ala-(R²)_{y} (SEQ ID NO:15) in which:
R¹ is an amino acid sequence comprising from 1 to about 40 amino acids wherein each amino acid is independently selected from the group consisting of naturally occurring amino acids and amino acid analogs;
R² is an amino acid sequence comprising from 1 to about 40 amino acids wherein each amino acid is independently selected from the group consisting of naturally occurring amino acids and amino acid analogs;
and x and y are independently selected and are equal to zero or one.

29. The method of claim 28, wherein the ADNF I polypeptide is Ser-Ala-Leu-Leu-Arg-Ser-Ile-Pro-Ala (SEQ ID NO: 13).

30.-31. (canceled)

32. The method of claim 24, wherein the ADNF polypeptide is an ADNF III polypeptide.

33. The method of claim 32, wherein the active core site of the ADNF III polypeptide comprises at least one D-amino acid.

34. The method of claim 32, wherein the ADNF III polypeptide has the formula (R¹)_{X}Asn-Ala-Pro-Val-Ser-Ile-Pro-Gln-(R²)_{y} (SEQ ID NO:22) in which:
R¹ is an amino acid sequence comprising from 1 to about 40 amino acids wherein each amino acid is independently selected from the group consisting of naturally occurring amino acids and amino acid analogs;
R² is an amino acid sequence comprising from 1 to about 40 amino acids wherein each amino acid is independently selected from the group consisting of naturally occurring amino acids and amino acid analogs;
and x and y are independently selected and are equal to zero or one.

35. The method of claim 34, wherein the ADNF III polypeptide is Asn-Ala-Pro-Val-Ser-Ile-Pro-Gln (SEQ ID NO:14).

36.-37. (canceled)

38. The method of claim 24, wherein a mixture of the ADNF I polypeptide of part (a) and the ADNF III polypeptide of part (b) are administered to the subject.

39. The method of claim 38, wherein either or both active core sites of the ADNF I polypeptide and the ADNF III polypeptide comprise at least one D-amino acid.

40. The method of claim 38, wherein the ADNF I polypeptide is Ser-Ala-Leu-Leu-Arg-Ser-Ile-Pro-Ala (SEQ ID NO: 13), and wherein the ADNF III polypeptide is Asn-Ala-Pro-Val-Ser-Ile-Pro-Gln (SEQ ID NO:14).

41.-50. (canceled)
